(19) United States
(12) Patent Application Publication
(10) Pub. No.: US 2022/0387493 A1
(43) Pub. Date: Dec. 8, 2022

(54) METHOD FOR PRODUCING CYTOTOXIC EFFECTOR MEMORY T-CELLS FOR CAR T-CELL TREATMENT OF CANCER

(71) Applicant: BAYLOR COLLEGE OF MEDICINE, Houston, TX (US)

(72) Inventors: Vanaja KONDURI, Houston, TX (US); William K. DECKER, Houston, TX (US); Matthew M. HALPERT, Houston, TX (US); Meenakshi G. HEGDE, Houston, TX (US); Nabil M. AHMED, Houston, TX (US); Sujith K. JOSEPH, Houston, TX (US)

(73) Assignee: BAYLOR COLLEGE OF MEDICINE, Houston, TX (US)

(21) Appl. No.: 17/774,848

(22) PCT Filed: Nov. 6, 2020
(86) PCT No.: PCT/US2020/059344

§ 371 Date: May 5, 2022

Related U.S. Application Data
(60) Provisional application No. 62/931,670, filed on Nov. 6, 2019.

(51) Int. Cl. A61K 35/17; C07K 14/725; C12N 5/0783; A61K 39/00

(52) U.S. Cl. CPC A61K 35/17; C07K 14/7051; C12N 5/0636; A61K 39/0011; A61K 2039/5156; C07K 2319/03; C12N 2510/00

(57) ABSTRACT

Provided herein are methods of expanding CD161⁺ T cells. Also provided are methods and compositions for generating modified CD161₊ T cells comprising a chimeric antigen receptor (CAR). In particular aspects, CAR-expressing T cells are produced, expanded, and/or used in disease (e.g, cancer) treatments.

### REFERENCE TO RELATED APPLICATIONS

The present application claims the priority benefit of U.S. provisional application No. 62/931,670, filed Nov. 6, 2019, the entire contents of which is incorporated herein by reference.

### STATEMENT REGARDING FEDERALLY SPONSORED RESEARCH

This invention was made with government support under Grant No. AI127387 awarded by the National Institutes of Health. The government has certain rights in the invention.

### BACKGROUND

### 1. Field

The present disclosure relates generally to the fields of medicine, immunology, cell biology, and molecular biology. In certain aspects, the field of the disclosure concerns immunotherapy. More particularly, it concerns the generation of improved chimeric antigen receptor (CAR) T cells and therapeutic methods using such cells.

### 2. Description of Related Art

Pancreatic Ductal Adenocarcinoma (PDAC) is a highly aggressive tumor with an abysmal five-year survival rate of <9% (Ansari et al., 2015) despite aggressive surgery, radiation, and high-dose chemotherapy. In recent years, adoptive chimeric antigen receptor (CAR) T-cell therapy has shown great potential as a therapeutic modality in cancer, specifically for select CD19⁺ malignancies (Maude et al., 2018; Neelapu et al., 2017). The CAR construct consists of a single-chain fragment variable region (scFv) that targets cell surface tumor antigen, a transmembrane domain, a hinge region, and the intracellular signaling domain of CD3ζ typically fused to those of either 4-1BB or CD28 costimulatory molecules (van der Stegen et al., 2015). In a pilot phase I clinical trial, adoptive cell therapy of autologous, mesothelin-specific CAR-T cells were shown to be safe and mildly efficacious against chemotherapy-refractory metastatic human PDAC in a small number of patients (Beatty et al., 2018); however, CAR T cell therapy against pancreatic tumors is still poorly-evolved. Indeed, at present, few CAR-based therapies have demonstrated any significant efficacy in the solid tumor setting.

A critical characteristic of cell-mediated immunity to viral infection is the establishment of long-lived memory T-cell populations that provide durable immunity to subsequent challenge through accelerated expansion and cytotoxicity kinetics (Seaman et al., 2004). Several groups have previously identified an intriguing subset of such memory T cells (Martin et al., 2009; Turtle et al., 2009; Northfield et al., 2008; Takahashi et al., 2006; Assarsson et al., 2000; Billerbeck et al., 2010; Fergusson et al., 2011; Fergusson et al., 2016; Fergusson et al., 2014) that may be identified by expression of the natural cytotoxicity receptor NK1.1 in mice or CD161 in humans. In contrast to TCR invariant or CD8αα⁺CD161⁺ cells, the polyclonal αβ cell population exhibits a stem cell like capacity for self-renewal and differentiation, a distinct transcriptional profile with significant upregulation of genes from the granzyme super family (Fergusson et al., 2011; Fergusson et al., 2014), a characteristic anti-viral specificity (Fergusson et al., 2008; Billerbeck et al., 2010; Havenith et al., 2012; Neelapu et al., 2005), and tissue-homing properties (Billerbeck et al., 2010). Canonically, CD161 is known as an innate NK cell receptor but may also be expressed on CD4, CD8, and NKT cells (Fergusson et al., 2016). Though also found in circulation, CD8⁺CD161⁺ cells contribute to tissue pathogenesis during chronic viral infection and autoimmune conditions due to tissue-resident properties and/or a propensity for extravasation (Assarsson et al., 2000; Billerbeck et al., 2010; Annibali et al., 2011). Further, high expression levels of CD161 in tumor resident immune infiltrates are associated with substantially improved clinical outcome and survival in NSCLC (Braud et al., 2018).

### SUMMARY

In a first embodiment there is provided an in vitro or ex vivo method comprising: (a) obtaining a sample of cells, the sample comprising CD161⁺ T cells; and (b) culturing the T cells in the presence of IL-7, IL-15, and IL-21, thereby providing a population of T cells that are expanded in the number of CD161⁺ cells as compared to non-CD161⁺ cells. In certain aspects, the T cells comprise CD8⁺CD161⁺ T cells. In further aspects, the T cells comprise CD4⁺CD161⁺ T cells.

IL-7 may be present at about 5-20 ng/ml, IL-15 may be present at about 2.5-10 ng/ml, and/or IL-21 may be present at about 20-40 ng/ml, such as 10 ng/ml IL-7, 5 ng/ml IL-15, and/or 30 ng/ml IL-21. The method may further comprise purifying or enriching T cells for the presence of CD8⁺CD161⁺ cells in the sample prior to step (b). The method may further comprise purifying or enriching T cells for the presence of CD8⁺CD161⁺ cells in the sample after step (b). The enriching of T-cells in the sample may comprise fluorescent cell sorting, magnetic or paramagnetic bead separation. The culturing may persist for up to 7, 14, 21, 28, 35 or 42 days.

In some aspects, the cells are further cultured in a media comprising a CD3 and/or CD28 stimulating agent. In some aspects, the CD3 and/or CD28 stimulating agent comprises a CD3 and/or CD28-binding antibody. In some aspects, the cells are further cultured in a media comprising a CD3, CD28, and/or CD161 stimulating agent. In some aspects, the CD3, CD28, and/or CD161 stimulating agent comprises a CD3, CD28, and/or CD161-binding antibody. In some aspects, the cells are further cultured in a media comprising a CD3-binding antibody, a CD28-binding antibody, Clec2d, and/or a CD161-stimulating antibody. In some aspects, the cells are further cultured in a media comprising about 0.1 to 5.0, 0.3 to 3.0, or 0.5 to 2.0 μg/ml of a CD3-binding antibody, a CD28-binding antibody, Clec2d, and/or a CD161-stimulating antibody.

In one aspect, CD8⁺CD161⁺ cells, CD8⁺CD161_{neg} cells, and bulk PBMC are stimulated with plate bound anti-CD3/CD28 and expanded in a cytokine cocktail comprised of 10 ng/ml IL-7, 5 ng/ml IL-15, and 30 ng/ml IL-21 (all from Peprotech, Rocky Hill, N.J.). In one aspect, CD8⁺CD161⁺ cells are isolated cultured against stimulation with anti-CD/CD28/CD161 each at 1 ug/mL, and expanded in RPMI-1640, 10% FBS, and 2 mmol/1 GlutaMAX in a cytokine cocktail comprised of 10 ng/ml IL-7, 5 ng/ml IL-15, and 30 ng/ml IL-21. The cells are placed in a humified chamber at 37° C. for 48 hours. After 48 hours, the cells are expanded with the IL7/15/21 cytokine cocktails without antibody stimulation.

The method may further comprise obtaining the cells from a subject, such as by obtained by apheresis or venipuncture. The sample may be a cryopreserved sample. The sample may be from umbilical cord blood. The sample may be a peripheral blood sample from the subject. The sample may comprise a subpopulation of T cells comprising an increased percentage of CD8⁺CD161⁺ cells as compared to a comparable sample as obtained from the subject. The sample may be obtained from a 3^{rd} party.

The method may further comprise introducing a nucleic acid encoding a CAR into a T cell in said sample, such as with a viral vector or through method not involving transducing the T cell with virus. Introducing the nucleic acid encoding a CAR or transgenic TCR into the T cell may occur prior to step (b) or after to step (b). The T cell may be inactivated for expression of an endogenous T-cell receptor and/or endogenous HLA.

The method may further comprise introducing a nucleic acid encoding a membrane-bound Cγ cytokine into the T cell, such as where the membrane-bound Cγ cytokine is a membrane bound IL-15. The membrane-bound Cγ cytokine may be an IL-15-IL-15Rα fusion protein.

The culturing may comprise culturing the T cells in the presence of dendritic cells or artificial antigen presenting cells (aAPCs). The aAPCs may comprise a CAR-binding or transgenic TCR-binding antibody or fragment thereof expressed on the surface of the aAPCs. The aAPCs may comprise additional molecules that activate or co-stimulate T cells. The additional molecules may comprise membrane-bound Cγ cytokines. The culturing of T cells in the presence of aAPCs may comprise culturing the cells at a ratio of about 10:1 to about 1:10 (CAR cells to aAPCs).

The method may further comprise cryopreserving a sample of the population of transgenic CAR or transgenic TCR cells. The CAR or transgenic TCR may be targeted to a cancer-cell antigen, such as CD19, CD20, ROR1, CD22carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gp120, HIV-1 envelope glycoprotein gp41, GD2, CD123, CD33, CD138, CD23, CD30, CD56, c-Met, meothelin, GD3, HERV-K, IL-11Rα, κ chain, λ chain, CSPG4, ERBB2, EGFRvIII, VEGFR2, HER2-HER3 in combination, or HER1-HER2 in combination. The CAR or transgenic TCR may be targeted to a pathogen antigen, such as a fungal, viral, or bacterial pathogen. The pathogen may be a Plasmodium, trypanosome, Aspergillus, Candida, HSV, HIV, RSV, EBV, CMV, JC virus, BK virus, or Ebola pathogen.

The method may further comprise assessing CD8⁺CD161⁺ cell content of said sample prior to step (b), after step (b), or both before and after step (b), such as by cell counting/flow cytometry.

Also provided is a T-cell composition made by a method as described herein.

A further embodiment involves a method of providing a T-cell response in a human subject having a disease comprising administering an effective amount of T cells as described herein. The disease may be a cancer and wherein the CAR or transgenic TCR is targeted to a cancer cell antigen. The subject may have undergone a previous anti-cancer therapy. The subject may be in remission or be free of symptoms of the cancer but comprises detectable cancer cells.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Gene expression analysis by microarray of antigenically stimulated T cells indicated significant upregulation of cytotoxic and innate-like characteristics of the CD8⁺NK1.1⁺ cells. A cohort of 15 mice received dendritic cell-based vaccination in combination with chemotherapy against murine pancreatic ductal adenocarcinoma. 60 days post tumor inoculation, spleens were harvested, pooled into three groups of five each and activated overnight with dendritic cells loaded with tumor antigens. Antigenically stimulated cells were then sorted by flow cytometry into NK1.1^{neg} and NK1.1⁺ subsets by gating on CD8⁺CD69⁺ population. Volcano plot shows 1642 genes that are significantly regulated between CD8⁺NK1.1^{neg} and CD8⁺NK1.1⁺ cells at a univariate significance level of 0.1. Top 15 genes that are differentially regulated at an FDR of 0.05 are labeled on the plot.

FIGS. 2A-F. CD8⁺NK1.1⁺ cells define a memory population that offers durable protection and improves survival against influenza infections and melanoma tumors. In the influenza model, splenocytes were harvested from mice upon recovery following influenza infection and sorted into CD8⁺NK1.1^{neg} and CD8⁺NK1.1⁺ cells and adoptively transferred into naïve mice that were subsequently challenged with influenza. In the melanoma model, tumor bearing mice were vaccinated with dendritic cells loaded with tumor antigens. Splenocytes were harvested after three weeks and sorted into CD8⁺NK1.1^{neg} and CD8⁺NK1.1⁺ cells and adoptively transferred into mice with palpable tumors. Adoptive transfer of antigen-experienced CD8⁺NK1.1⁺ cells offered durable protection against influenza infections (FIGS. 2A-C) and melanoma tumors (FIGS. 2D-F). (FIG. 2A) Mice that received CD8⁺NK1.1⁺ cells recovered their body weight upon influenza infection compared to mice that received CD8⁺NK1.1^{neg} and naïve CD8⁺ cells. (FIG. 2B) One hundred percent survival was seen in mice that received CD8⁺NK1.1⁺ cells compared to the groups that received CD8⁺NK1.1^{neg} and naïve CD8⁺ cells. (FIG. 2C) Analysis of PBMC two weeks post infection showed a 40% increase in circulating CD3⁺CD8⁺IFN-γ⁺ cells (p<0.003) among mice that received CD8⁺NK1.1⁺ cells in comparison to naïve and CD8⁺NK1.1^{neg} adoptively transferred cohorts. (FIGS. 2D-E) In the melanoma model, mice that received CD8⁺NK1.1⁺ cells exhibited delayed tumor growth and improved survival. (FIG. 2F) Analysis of peripheral blood lymphocytes three weeks post tumor implantation indicated significantly elevated levels of memory markers CD62L and CCR7 among GP100 tetramer-specific CD8⁺ cells in the cohort adoptively transferred with CD8⁺NK1.1⁺ cells in comparison to cohorts adoptively transferred with CD8⁺NK1.1^{neg} or naïve splenocytes. For each experiment, n=10 mice per group. Error bars=+/−SEM, *p<0.05, one-way ANOVA.

FIG. 3. Murine CD3⁺CD8⁺NK1.1⁺ cell population is phenotypically preserved among human CD3⁺CD8⁺CD161⁺ Counterparts. CD3⁺CD8⁺CD161⁺ and CD3⁺CD8⁺CD161^{neg} cells, human equivalents of the murine CD3⁺CD8⁺NK1.1⁺ cells and CD3⁺CD8⁺NK1.1^{neg} cells were magnetically sorted from the peripheral blood of six human donors and gene expression profile analysis was performed by microarray. Volcano plot showing the differential regulation of genes between CD8⁺CD161⁺ and CD8⁺CD161^{neg} cells highlights CD161 receptor upregulation in the oval.

FIG. 4. CD8+CD161+, CD8+CD161neg, and unmanipulated bulk PBMC were freshly isolated from a human peripheral blood product. Isolated cells were immediately tested for cytotoxic capacity in a four-hour killing assay using ⁵¹Cr-labeled allogeneic 293-HEK targets. As shown, CD8⁺CD161⁺ cells could induce 100% target lysis at an E:T ratio of 25:1 whereas bulk PBMC and CD8⁺CD161^{neg} cells exhibited lytic capabilities of 22% and 15%, respectively, at the maximum E:T ratio of 50:1 (p<0.002 at 50:1, p<0.0007 at 25:1, and p<0.00002 at 5:1 by One-way ANOVA). X-axis—E:T ratio. Y-axis—percent killing. Error bars=+/−SD.

FIG. 5. Ex vivo expansion of CD8⁺CD161⁺ cells with IL7/15/21 in combination with plate bound stimulation with anti-CD3/CD28/Clec2d enhanced the central memory phenotype (CD45RA⁻CCR7⁺). CD8⁺CD161⁺ cells were sorted from normal donor and ex vivo stimulation conditions were optimized. Cells are not CAR transduced. Compared to IL2, IL-2/7/15, IL2/7/15/21 stimulation, a combination of IL7/15/21 with plate bound stimulation of anti-CD3/CD28/Clec2d resulted in significant upregulation of central memory (CD45RA⁻CCR7⁺).

FIG. 6. Ex vivo expansion of CD8⁺CD161⁺ cells with IL7/15/21 in combination with plate bound stimulation with anti-CD3/CD28/Clec2d enhanced the cytotoxic granzyme production. CD8⁺CD161⁺ cells were sorted from normal donor and ex vivo stimulation conditions were optimized. Cells are not CAR transduced. Compared to IL2, IL-2/7/15, IL2/7/15/21 stimulation, a combination of IL7/15/21 with plate bound stimulation of anti-CD3/CD28/Clec2d resulted in significant upregulation of cytotoxic molecules, granzyme and perforin.

FIGS. 7A-B. CD8⁺NK1.1⁺ cells are identified as critical circulating memory cells in multiple mouse models of disease. To validate that the protective effects of CD8⁺NK1.1⁺ cells are model-independent, adoptive transfer experiments of the CD8⁺NK1.1⁺ cells were performed in influenza infection model and melanoma tumor model. (FIG. 7A) Naïve mice were exposed to sublethal-dose of influenza, mice were allowed to recover from infection and three weeks post infection, splenocytes were harvested and magnetically sorted into CD8⁺NK1.1^{neg} and CD8⁺NK1.1⁺ cells. 5×10⁵ cells/mouse of each NK1.1 group were adoptively transferred into naïve cohorts that were lethally challenged with the same influenza virus strain 24 hours after adoptive transfer. Mice that received naïve CD8⁺ splenocytes served as controls. (FIG. 7B) Naïve mice were inoculated sub-cutaneously with 2×10⁵B16 melanoma cells and vaccinated with a B16-antigen loaded cell-based vaccine at 7- and 14-days post-inoculation. At day 21, mice were sacrificed, and splenocytes harvested and sorted into CD8⁺NK1.1^{neg} and CD8⁺NK1.1⁺ cell populations. Naïve cohorts inoculated with palpable B16 tumors were then each adoptively transferred with 1.5×10⁶ CD8⁺NK1.1^{neg} and CD8⁺NK1.1⁺ cells by intraperitoneal injection. Mice that received naïve CD8⁺ splenocytes served as controls.

FIG. 8. TCR-Vβ spectratyping indicated CD3⁺CD8⁺CD161⁺ cells as polyclonal in nature. To confirm the clonal nature of the CD8⁺CD161⁺ cells, TCR-Vβ spectratyping was performed on the donor derived cells. Histograms from the amplification of 30 TCR Vβ families were unskewed exhibiting a Gaussian distribution of the CDR3 size suggesting polyclonal nature of these cells.

FIG. 9. Cross-species comparative gene analysis reveals a conserved gene signature of 206 genes differentially regulated between the two populations. 206 common genes were identified between the mouse (15 pooled samples) and human (6 paired samples) microarray analysis based on nomenclature. The expression pattern for these genes was similar between activated CD8⁺NK1.1⁺ cells and resting CD8⁺CD161⁺ cells indicating the highly conserved nature of the gene signature.

### DESCRIPTION OF ILLUSTRATIVE EMBODIMENTS

As discussed above, CAR-T therapies are showing great promise in the treatment of cancers such as metastatic murine ductal adenocarcinoma (PDAC). In previous work, the inventors demonstrated that adoptively transferred, antigen experienced CD8⁺NK1.1⁺ cells could mediate durable protection in a model of PDAC. Interestingly, these cells were present nine months after initial exposure to antigen and were highly protective when adoptively transferred into naïve mice subsequently challenged with the parent PDAC cell line (Konduri et al., 2016). Expanding upon these results, the inventors sought to characterize additional biological and functional properties of these cells in a variety of in vivo model systems including a SCID xenograft model of CAR T-cell therapy for the treatment of PDAC. The results demonstrated that CD8⁺CD161⁺ T cells comprise a superior platform for CAR T-cell therapy provided that accommodation is made to prevent differentiation of the starting cell population during transduction and expansion. Moreover, they have now developed an improved method by which such cells can be expanded ex vivo, thereby making it easier to provide CAR-T therapies to subjects in need thereof. These and other features of the disclosure are set out in greater detail below.

### I. DEFINITIONS

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

The term “chimeric antigen receptors (CARs),” as used herein, may refer to artificial T-cell receptors, chimeric T-cell receptors, transgenic T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. In some embodiments, CARs comprise an intracellular activation domain, a transmembrane domain, and an extracellular domain comprising a tumor associated antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3-zeta, FcR, CD27, CD28, CD137, DAP10, and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

The term “T-cell receptor (TCR)” as used herein refers to a protein receptor on T cells that is composed of a heterodimer of an alpha (α) and beta (β) chain, although in some cells the TCR consists of gamma and delta (γ/δ) chains. In embodiments of the disclosure, the TCR may be modified on any cell comprising a TCR, including a helper T cell, a cytotoxic T cell, a memory T cell, regulatory T cell, natural killer T cell, and gamma delta T cell, for example.

The terms “tumor-associated antigen” and “cancer cell antigen” are used interchangeably herein. In each case, the terms refer to proteins, glycoproteins or carbohydrates that are specifically or preferentially expressed by cancer cells.

### II. CHIMERIC ANTIGEN RECEPTORS

As used herein, the term “antigen” is a molecule capable of being bound by an antibody or T-cell receptor. An antigen is additionally capable of inducing a humoral immune response and/or cellular immune response leading to the production of B and/or T lymphocytes.

Embodiments of the present disclosure involve nucleic acids, including nucleic acids encoding an antigen-specific chimeric antigen receptor (CAR) polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprised of the shared space between one or more antigens. Pattern recognition receptors, such as Dectin-1, may be used to derive specificity to a carbohydrate antigen. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor. A complementarity determining region (CDR) is a short amino acid sequence found in the variable domains of antigen receptor (e.g., immunoglobulin and T-cell receptor) proteins that complements an antigen and therefore provides the receptor with its specificity for that particular antigen. Each polypeptide chain of an antigen receptor contains three CDRs (CDR1, CDR2, and CDR3). Since the antigen receptors are typically composed of two polypeptide chains, there are six CDRs for each antigen receptor that can come into contact with the antigen—each heavy and light chain contains three CDRs. Because most sequence variation associated with immunoglobulins and T-cell receptors are found in the CDRs, these regions are sometimes referred to as hypervariable domains. Among these, CDR3 shows the greatest variability as it is encoded by a recombination of the VJ (VDJ in the case of heavy chain and TCR αβ chain) regions.

It is contemplated that the human CAR nucleic acids are human genes to enhance cellular immunotherapy for human patients. In a specific embodiment, the disclosure includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into what has been referred to by Winters as a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8α.

The intracellular signaling domain of the chimeric receptor of the disclosure is responsible for activation of at least one of the normal effector functions of the immune cell in which the chimeric receptor has been placed. The term “effector function” refers to a specialized function of a differentiated cell. Effector function of a T cell, for example, may be cytolytic activity or helper activity including the secretion of cytokines. Effector function in a naive, memory, or memory-type T cell includes antigen-dependent proliferation. Thus, the term “intracellular signaling domain” refers to the portion of a protein that transduces the effector function signal and directs the cell to perform a specialized function. While usually the entire intracellular signaling domain will be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term intracellular signaling domain is thus meant to include any truncated portion of the intracellular signaling domain sufficient to transduce the effector function signal. Examples include the zeta chain of the T-cell receptor or any of its homologs (e.g., eta, delta, gamma, or epsilon), MB1 chain, B29, Fc RIII, Fc RI, and combinations of signaling molecules, such as CD3ζ and CD28, CD27, 4-1BB, DAP-10, OX40, and combinations thereof, as well as other similar molecules and fragments. Intracellular signaling portions of other members of the families of activating proteins can be used, such as FcγRIII and FcεRI. In a preferred embodiment, the human CD3 ζ intracellular domain was taken for activation.

The antigen-specific extracellular domain and the intracellular signaling-domain may be linked by a transmembrane domain, such as the human IgG₄Fc hinge and Fc regions. Alternatives include the human CD4 transmembrane domain, the human CD28 transmembrane domain, the transmembrane human CD3ζ domain, or a cysteine mutated human CD3ζ domain, or other transmembrane domains from other human transmembrane signaling proteins, such as CD16 and CD8 and erythropoietin receptor.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, and 4-1BB (CD137). In addition to a primary signal initiated by CD3 ζ, an additional signal provided by a human costimulatory receptor inserted in a human CAR is important for full activation of T cells and could help improve in vivo persistence and the therapeutic success of the adoptive immunotherapy.

In particular embodiments, the disclosure concerns isolated nucleic acid segments and expression cassettes incorporating DNA sequences that encode the CAR. Vectors of the present disclosure are designed, primarily, to deliver desired genes to immune cells, preferably T cells under the control of regulated eukaryotic promoters, for example, MNDU3 promoter, CMV promoter, EF1α promoter, or Ubiquitin promoter. Also, the vectors may contain a selectable marker, if for no other reason, to facilitate their manipulation in vitro. In other embodiments, the CAR can be expressed from mRNA in vitro transcribed from a DNA template.

Chimeric antigen receptor molecules are recombinant and are distinguished by their ability to both bind antigen and transduce activation signals via immunoreceptor activation motifs (ITAM's) present in their cytoplasmic tails. Receptor constructs utilizing an antigen-binding moiety (for example, generated from single chain antibodies (scFv)) afford the additional advantage of being “universal” in that they bind native antigen on the target cell surface in an HLA-independent fashion. For example, several laboratories have reported on scFv constructs fused to sequences coding for the intracellular portion of the CD3 complex's zeta chain (ζ), the Fc receptor gamma chain, and sky tyrosine kinase (Eshhar et al., 1993; Fitzer-Attas et al., 1998). Re-directed T cell effector mechanisms including tumor recognition and lysis by CTL have been documented in several murine and human antigen-scFv: ζ systems (Eshhar, 1997; Altenschmidt et al., 1997; Brocker et al., 1998).

To date non-human antigen binding regions are typically used in constructing a chimeric antigen receptor. A potential problem with using non-human antigen binding regions, such as murine monoclonal antibodies, is the lack of human effector functionality and inability to penetrate into tumor masses. In other words, such antibodies may be unable to mediate complement-dependent lysis or lyse human target cells through antibody-dependent cellular toxicity or Fc-receptor mediated phagocytosis to destroy cells expressing CAR. Furthermore, non-human monoclonal antibodies can be recognized by the human host as a foreign protein, and therefore, repeated injections of such foreign antibodies can lead to the induction of immune responses leading to harmful hypersensitivity reactions. For murine-based monoclonal antibodies, this is often referred to as a Human Anti-Mouse Antibody (HAMA) response. Therefore, the use of human antibodies is more preferred because they do not elicit as strong a HAMA response as murine antibodies. Similarly, the use of human sequences in the CAR can avoid immune-mediated recognition and therefore elimination by endogenous T cells that reside in the recipient and recognize processed antigen in the context of HLA.

In some embodiments, the chimeric antigen receptor comprises: a) an intracellular signaling domain, b) a transmembrane domain, and c) an extracellular domain comprising an antigen binding region.

In specific embodiments, intracellular receptor signaling domains in the CAR include those of the T cell antigen receptor complex, such as the zeta chain of CD3, also Fcγ RIII costimulatory signaling domains, CD28, CD27, DAP10, CD137, OX40, CD2, alone or in a series with CD3zeta, for example. In specific embodiments, the intracellular domain (which may be referred to as the cytoplasmic domain) comprises part or all of one or more of TCR zeta chain, CD28, CD27, OX40/CD134, 4-1BB/CD137, FccRIγ, ICOS/CD278, IL-2Rbeta/CD122, IL-2Ra/CD132, DAP10, DAP12, and CD40. In some embodiments, one employs any part of the endogenous T cell receptor complex in the intracellular domain. One or multiple cytoplasmic domains may be employed, as so-called third generation CARs have at least two or three signaling domains fused together for additive or synergistic effect, for example.

In certain embodiments of the chimeric antigen receptor, the antigen-specific portion of the receptor (which may be referred to as an extracellular domain comprising an antigen binding region) comprises a tumor associated antigen or a pathogen-specific antigen binding domain including carbohydrate antigen recognized by pattern-recognition receptors, such as Dectin-1. A tumor associated antigen may be of any kind so long as it is expressed on the cell surface of tumor cells. Exemplary embodiments of tumor associated antigens include CD19, CD20, carcinoembryonic antigen, α-fetoprotein, CA-125, MUC-1, CD56, EGFR, c-Met, AKT, Her2, Her3, epithelial tumor antigen, melanoma-associated antigen, mutated p53, mutated ras, and so forth. In certain embodiments, the CAR can be co-expressed with a membrane-bound cytokine to improve persistence when there is a low amount of tumor-associated antigen. For example, CAR can be co-expressed with membrane-bound IL-15.

In certain embodiments intracellular tumor associated antigens may be targeted, such as HA-1, survivin, WT1, and p53. This can be achieved by a CAR expressed on a universal T cell that recognizes the processed peptide described from the intracellular tumor associated antigen in the context of HLA. In addition, the universal T cell may be genetically modified to express a T-cell receptor pairing that recognizes the intracellular processed tumor associated antigen in the context of HLA.

The pathogen may be of any kind, but in specific embodiments the pathogen is a fungus, bacteria, or virus, for example. Exemplary viral pathogens include those of the families of Adenoviridae, Epstein-Barr virus (EBV), Cytomegalovirus (CMV), Respiratory Syncytial Virus (RSV), JC virus, BK virus, HSV, HHV family of viruses, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae. Exemplary pathogenic viruses cause smallpox, influenza, mumps, measles, chickenpox, ebola, and rubella. Exemplary pathogenic fungi include Candida, Aspergillus, Cryptococcus, Histoplasma, Pneumocystis, and Stachybotrys. Exemplary pathogenic bacteria include Streptococcus, Pseudomonas, Shigella, Campylobacter, Staphylococcus, Helicobacter, E. coli, Rickettsia, Bacillus, Bordetella, Chlamydia, Spirochetes, and Salmonella. In one embodiment the pathogen receptor Dectin-1 can be used to generate a CAR that recognizes the carbohydrate structure on the cell wall of fungi. T cells genetically modified to express the CAR based on the specificity of Dectin-1 can recognize Aspergillus and target hyphal growth. In another embodiment, CARs can be made based on an antibody recognizing viral determinants (e.g., the glycoproteins from CMV and Ebola) to interrupt viral infections and pathology.

In some embodiments, the pathogenic antigen is an Aspergillus carbohydrate antigen for which the extracellular domain in the CAR recognizes patterns of carbohydrates of the fungal cell wall, such as via Dectin-1.

A chimeric immunoreceptor according to the present disclosure can be produced by any means known in the art, though preferably it is produced using recombinant DNA techniques. A nucleic acid sequence encoding the several regions of the chimeric receptor can be prepared and assembled into a complete coding sequence by standard techniques of molecular cloning (genomic library screening, PCR, primer-assisted ligation, scFv libraries from yeast and bacteria, site-directed mutagenesis, etc.). The resulting coding region can be inserted into an expression vector and used to transform a suitable expression host allogeneic T-cell line.

As used herein, a nucleic acid construct or nucleic acid sequence or polynucleotide is intended to mean a DNA molecule that can be transformed or introduced into a T cell and be transcribed and translated to produce a product (e.g., a chimeric antigen receptor).

In an exemplary nucleic acid construct (polynucleotide) employed in the present disclosure, the promoter is operably linked to the nucleic acid sequence encoding the chimeric receptor of the present disclosure, i.e., they are positioned so as to promote transcription of the messenger RNA from the DNA encoding the chimeric receptor. The promoter can be of genomic origin or synthetically generated. A variety of promoters for use in T cells are well-known in the art (e.g., the CD4 promoter disclosed by Marodon et al., 2003). The promoter can be constitutive or inducible, where induction is associated with the specific cell type or a specific level of maturation, for example. Alternatively, a number of well-known viral promoters are also suitable. Promoters of interest include the β-actin promoter, SV40 early and late promoters, immunoglobulin promoter, human cytomegalovirus promoter, retrovirus promoter, and the Friend spleen focus-forming virus promoter. The promoters may or may not be associated with enhancers, wherein the enhancers may be naturally associated with the particular promoter or associated with a different promoter.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA or provide T cell-specific expression (Barthel and Goldfeld, 2003). Also, it may be further advantageous to use endogenous or exogenous non-coding regions to stabilize the mRNA.

For expression of a chimeric antigen receptor of the present disclosure, the naturally occurring or endogenous transcriptional initiation region of the nucleic acid sequence encoding N-terminal components of the chimeric receptor can be used to generate the chimeric receptor in the target host. Alternatively, an exogenous transcriptional initiation region can be used that allows for constitutive or inducible expression, wherein expression can be controlled depending upon the target host, the level of expression desired, the nature of the target host, and the like.

Likewise, a signal sequence directing the chimeric receptor to the surface membrane can be the endogenous signal sequence of N-terminal component of the chimeric receptor. Optionally, in some instances, it may be desirable to exchange this sequence for a different signal sequence. However, the signal sequence selected should be compatible with the secretory pathway of T cells so that the chimeric receptor is presented on the surface of the T cell.

Similarly, a termination region may be provided by the naturally occurring or endogenous transcriptional termination region of the nucleic acid sequence encoding the C-terminal component of the chimeric receptor. Alternatively, the termination region may be derived from a different source. For the most part, the source of the termination region is generally not considered to be critical to the expression of a recombinant protein and a wide variety of termination regions can be employed without adversely affecting expression.

As will be appreciated by one of skill in the art that, in some instances, a few amino acids at the ends of the antigen binding domain in the CAR can be deleted, usually not more than 10, more usually not more than 5 residues, for example. Also, it may be desirable to introduce a small number of amino acids at the borders, usually not more than 10, more usually not more than 5 residues. The deletion or insertion of amino acids may be as a result of the needs of the construction, providing for convenient restriction sites, ease of manipulation, improvement in levels of expression, or the like. In addition, the substitute of one or more amino acids with a different amino acid can occur for similar reasons, usually not substituting more than about five amino acids in any one domain.

The chimeric construct that encodes the chimeric receptor according to the disclosure can be prepared in conventional ways. Because, for the most part, natural sequences may be employed, the natural genes may be isolated and manipulated, as appropriate, so as to allow for the proper joining of the various components. Thus, the nucleic acid sequences encoding for the N-terminal and C-terminal proteins of the chimeric receptor can be isolated by employing the polymerase chain reaction (PCR), using appropriate primers that result in deletion of the undesired portions of the gene. Alternatively, restriction digests of cloned genes can be used to generate the chimeric construct. In either case, the sequences can be selected to provide for restriction sites that are blunt-ended or have complementary overlaps.

The various manipulations for preparing the chimeric construct can be carried out in vitro and in particular embodiments the chimeric construct is introduced into vectors for cloning and expression in an appropriate host using standard transformation or transfection methods. Thus, after each manipulation, the resulting construct from joining of the DNA sequences is cloned, the vector isolated, and the sequence screened to ensure that the sequence encodes the desired chimeric receptor. The sequence can be screened by restriction analysis, sequencing, or the like.

The chimeric constructs of the present disclosure find application in subjects having or suspected of having cancer by reducing the size of a tumor or preventing the growth or re-growth of a tumor in these subjects. Accordingly, the present disclosure further relates to a method for reducing growth or preventing tumor formation in a subject by introducing a chimeric construct of the present disclosure into an isolated T cell of the subject and reintroducing into the subject the transformed T cell, thereby effecting anti-tumor responses to reduce or eliminate tumors in the subject. Suitable T cells that can be used include cytotoxic lymphocytes (CTL) or any cell having a T cell receptor in need of disruption. As is well-known to one of skill in the art, various methods are readily available for isolating these cells from a subject. For example, using cell surface marker expression or using commercially available kits (e.g., ISOCELL™ from Pierce, Rockford, Ill.).

It is contemplated that the chimeric construct can be introduced into the subject's own T cells as naked DNA or in a suitable vector. Methods of stably transfecting T cells by electroporation using naked DNA are known in the art. See, e.g., U.S. Pat. No. 6,410,319. Naked DNA generally refers to the DNA encoding a chimeric receptor of the present disclosure contained in a plasmid expression vector in proper orientation for expression. Advantageously, the use of naked DNA reduces the time required to produce T cells expressing the chimeric receptor of the present disclosure.

Alternatively, a viral vector (e.g., a retroviral vector, adenoviral vector, adeno-associated viral vector, or lentiviral vector) can be used to introduce the chimeric construct into T cells. Suitable vectors for use in accordance with the method of the present disclosure are non-replicating in the subject's T cells. A large number of vectors are known that are based on viruses, where the copy number of the virus maintained in the cell is low enough to maintain the viability of the cell. Illustrative vectors include the pFB-neo vectors (STRATAGENE®) disclosed herein as well as vectors based on HIV, SV40, EBV, HSV, or BPV.

Once it is established that the transfected or transduced T cell is capable of expressing the chimeric receptor as a surface membrane protein with the desired regulation and at a desired level, it can be determined whether the chimeric receptor is functional in the host cell to provide for the desired signal induction. Subsequently, the transduced T cells are reintroduced or administered to the subject to activate anti-tumor responses in the subject. To facilitate administration, the transduced T cells according to the disclosure can be made into a pharmaceutical composition or made into an implant appropriate for administration in vivo, with appropriate carriers or diluents, which further can be pharmaceutically acceptable. The means of making such a composition or an implant have been described in the art (see, for instance, Remington's Pharmaceutical Sciences, 16th Ed., Mack, ed., 1980). Where appropriate, the transduced T cells can be formulated into a preparation in semisolid or liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. Desirably, however, a pharmaceutically acceptable form is employed that does not ineffectuate the cells expressing the chimeric receptor. Thus, desirably the transduced T cells can be made into a pharmaceutical composition containing a balanced salt solution, preferably Hanks' balanced salt solution, or normal saline.

### III. METHODS AND COMPOSITIONS RELATED TO THE EMBODIMENTS

In certain aspects, the disclosure includes a method of making and/or expanding the antigen-specific CD8⁺CD161⁺ T cells that comprises transfecting T cells with an expression vector containing a DNA construct encoding the hCAR, then, optionally, stimulating the cells with antigen positive cells, recombinant antigen, or an antibody to the receptor to cause the cells to proliferate. As described in the examples, a particular combination of interleukins—IL-7, IL-15 and IL-21—provide for substantially improved expansion of CD8⁺CD161⁺ T cells.

In another aspect, a method is provided of stably transfecting and re-directing T cells by electroporation, or other non-viral gene transfer (such as, but not limited to sonoporation) using naked DNA. Most investigators have used viral vectors to carry heterologous genes into T cells. By using naked DNA, the time required to produce redirected T cells can be reduced. “Naked DNA” means DNA encoding a chimeric T-cell receptor (cTCR) contained in an expression cassette or vector in proper orientation for expression. The electroporation method of this disclosure produces stable transfectants that express and carry on their surfaces the chimeric TCR (cTCR).

“Chimeric TCR” means a receptor that is expressed by T cells and that comprises intracellular signaling, transmembrane, and extracellular domains, where the extracellular domain is capable of specifically binding in an MHC unrestricted manner an antigen that is not normally bound by a T-cell receptor in that manner. Stimulation of the T cells by the antigen under proper conditions results in proliferation (expansion) of the cells and/or production of IL-2. The exemplary chimeric receptors of the instant application are examples of a chimeric TCR. However, the method is applicable to transfection with chimeric TCRs that are specific for other target antigens, such as chimeric TCRs that are specific for HER2/Neu (Stancovski et al., 1993), ERBB2 (Moritz et al., 1994), folate binding protein (Hwu et al., 1995), renal cell carcinoma (Weitjens et al., 1996), and HIV-1 envelope glycoproteins gp120 and gp41 (Roberts et al., 1994). Other cell-surface target antigens include, but are not limited to, CD20, carcinoembryonic antigen, mesothelin, ROR1, c-Met, CD56, GD2, GD3, α-fetoprotein, CD23, CD30, CD123, IL-11Rα, κ chain, λ chain, CD70, CA-125, MUC-1, EGFR and variants, epithelial tumor antigen, and so forth.

In certain aspects, the T cells are primary human T cells, such as T cells derived from human peripheral blood mononuclear cells (PBMC), PBMC collected after stimulation with G-CSF, bone marrow, or umbilical cord blood. Conditions include the use of mRNA and DNA and electroporation. Following transfection, the cells may be immediately infused or may be stored. In certain aspects, following transfection, the cells may be propagated for days, weeks, or months ex vivo as a bulk population within about 1, 2, 3, 4, 5 days or more following gene transfer into cells. In a further aspect, following transfection, the transfectants are cloned and a clone demonstrating presence of a single integrated or episomally maintained expression cassette or plasmid, and expression of the chimeric receptor is expanded ex vivo. The clone selected for expansion demonstrates the capacity to specifically recognize target cells. The recombinant T cells may be expanded by stimulation with IL-2, or other cytokines that bind the common gamma-chain (e.g., IL-7, IL-12, IL-15, IL-21, and others). The recombinant T cells may be expanded by stimulation with artificial antigen presenting cells. The recombinant T cells may be expanded on artificial antigen presenting cell or with an antibody, such as OKT3, which cross links CD3 on the T cell surface. Subsets of the recombinant T cells may be deleted on artificial antigen presenting cell or with an antibody, such as Campath, which binds CD52 on the T cell surface. In a further aspect, the genetically modified cells may be cryopreserved.

T-cell propagation (survival) after infusion may be assessed by (i) q-PCR using primers specific for the CAR; (ii) flow cytometry using an antibody specific for the CAR; and/or (iii) soluble TAA.

In certain embodiments of the disclosure, the CAR cells are delivered to an individual in need thereof, such as an individual that has cancer or an infection. The cells then enhance the individual's immune system to attack the respective cancer or pathogenic cells. In some cases, the individual is provided with one or more doses of the antigen-specific CAR T-cells. In cases where the individual is provided with two or more doses of the antigen-specific CAR T-cells, the duration between the administrations should be sufficient to allow time for propagation in the individual, and in specific embodiments the duration between doses is 1, 2, 3, 4, 5, 6, 7, or more days.

The source of the allogeneic T cells that are modified to include both a chimeric antigen receptor and that lack functional TCR may be of any kind, but in specific embodiments the cells are obtained from a bank of umbilical cord blood, peripheral blood, human embryonic stem cells, or induced pluripotent stem cells, for example. Suitable doses for a therapeutic effect would be at least 10⁵ or between about 10⁵ and about 10¹⁰ cells per dose, for example, preferably in a series of dosing cycles. An exemplary dosing regimen consists of four one-week dosing cycles of escalating doses, starting at least at about 10⁵ cells on Day 0, for example increasing incrementally up to a target dose of about 10¹⁰ cells within several weeks of initiating an intra-patient dose escalation scheme. Suitable modes of administration include intravenous, subcutaneous, intracavitary (for example by reservoir-access device), intraperitoneal, and direct injection into a tumor mass.

A pharmaceutical composition of the present disclosure can be used alone or in combination with other well-established agents useful for treating cancer. Whether delivered alone or in combination with other agents, the pharmaceutical composition of the present disclosure can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

A composition of the present disclosure can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the present disclosure, alone or in combination with other active agents, calculated in an amount sufficient to produce the desired effect, in association with a pharmaceutically acceptable diluent, carrier, or vehicle, where appropriate. The specifications for the novel unit dosage forms of the present disclosure depend on the particular pharmacodynamics associated with the pharmaceutical composition in the particular subject.

Desirably an effective amount or sufficient number of the isolated transduced T cells is present in the composition and introduced into the subject such that long-term, specific, anti-tumor responses are established to reduce the size of a tumor or eliminate tumor growth or regrowth than would otherwise result in the absence of such treatment. Desirably, the amount of transduced T cells reintroduced into the subject causes a 10%, 20%, 30%, 40%, 50%, 60%, 70%, 80%, 90%, 95%, 98%, or 100% decrease in tumor size when compared to otherwise same conditions wherein the transduced T cells are not present.

Accordingly, the amount of transduced T cells administered should take into account the route of administration and should be such that a sufficient number of the transduced T cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of transduced T cells desirably should be sufficient to provide in the subject being treated at least from about 1×10⁶ to about 1×10⁹ transduced T cells, even more desirably, from about 1×10⁷ to about 5×10⁸ transduced T cells, although any suitable amount can be utilized either above, e.g., greater than 5×10⁸ cells, or below, e.g., less than 1×10⁷ cells. The dosing schedule can be based on well-established cell-based therapies (see, e.g., Topalian and Rosenberg, 1987; U.S. Pat. No. 4,690,915), or an alternate continuous infusion strategy can be employed.

These values provide general guidance of the range of transduced T cells to be utilized by the practitioner upon optimizing the method of the present disclosure for practice of the disclosure. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in pharmacokinetics, drug disposition, and metabolism. One skilled in the art readily can make any necessary adjustments in accordance with the exigencies of the particular situation.

### IV. EXEMPLARY HUMAN ANTIGEN RECEPTOR T CELLS

As discussed above, the disclosure relates to the culturing and use of CD8⁺CD161⁺ T cells.

CD8 (cluster of differentiation 8) is a transmembrane glycoprotein that serves as a co-receptor for the T cell receptor (TCR). Like the TCR, CD8 binds to a major histocompatibility complex (MHC) molecule but is specific for the class I MHC protein. There are two isoforms of the protein, α and β, each encoded by a different gene. In humans, both genes are located on chromosome 2 in position 2p12.

The CD8 co-receptor is predominantly expressed on the surface of cytotoxic T cells, but can also be found on natural killer cells, cortical thymocytes, and dendritic cells. The CD8 molecule is a marker for cytotoxic T cell population. It is expressed in T cell lymphoblastic lymphoma and hypo-pigmented mycosis fungoides.

To function, CD8 forms a dimer, consisting of a pair of CD8 chains. The most common form of CD8 is composed of a CD8-α and CD8-β chain, both members of the immunoglobulin superfamily with an immunoglobulin variable (IgV)-like extracellular domain connected to the membrane by a thin stalk, and an intracellular tail. Less-common homodimers of the CD8-α chain are also expressed on some cells. The molecular weight of each CD8 chain is about 34 kDa. The structure of the CD8 molecule was determined by Leahy, D. J., Axel, R., and Hendrickson, W. A. by X-ray Diffraction at a 2.6 A resolution. The structure was determined to have an immunoglobulin-like beta-sandwich folding and 114 amino acid residues. 2% of the protein is wound into α-helices and 46% into β-sheets, with the remaining 52% of the molecules remaining in the loop portions.

The extracellular IgV-like domain of CD8-α interacts with the α₃ portion of the Class I MHC molecule. This affinity keeps the T cell receptor of the cytotoxic T cell and the target cell bound closely together during antigen-specific activation. Cytotoxic T cells with CD8 surface protein are called CD8+ T cells. The main recognition site is a flexible loop at the α₃ domain of an MHC molecule. This was discovered by doing mutational analyses. The flexible α₃ domain is located between residues 223 and 229 in the genome. In addition to aiding with cytotoxic T cell antigen interactions the CD8 co-receptor also plays a role in T cell signaling. The cytoplasmic tails of the CD8 co-receptor interact with Lck (lymphocyte-specific protein tyrosine kinase). Once the T cell receptor binds its specific antigen Lck phosphorylates the cytoplasmic CD3 and ζ-chains of the TCR complex which initiates a cascade of phosphorylation eventually leading to activation of transcription factors like NFAT, NF-κB, and AP-1 which affect the expression of certain genes.

CD161, also known as KLRB1 or NKR-P1A, is classified as a type II membrane protein because it has an external C terminus. CD161 recognizes Lectin Like Transcript-1 (LLT1) as a functional ligand. Natural killer (NK) cells are lymphocytes that mediate cytotoxicity and secrete cytokines after immune stimulation. Several genes of the C-type lectin superfamily, including the rodent NKRP1 family of glycoproteins, are expressed by NK cells and may be involved in the regulation of NK cell function. CD161 contains an extracellular domain with several motifs characteristic of C-type lectins, a transmembrane domain, and a cytoplasmic domain.

In one aspect compositions and methods of the embodiments concern human CD8⁺CD161⁺ T cell expressing a chimeric antigen receptor (or CAR) polypeptide). The CAR may be of any antigen binding specificity, but would comprise the typical intracellular signaling domain, transmembrane domain, and extracellular domain that are present in CAR constructs. The extracellular domain would comprise a given binding region depending on the use for which the CAR-T is designed. The binding region is a F(ab′)2, Fab′, Fab, Fv, or scFv. The binding region may comprise an amino acid sequence that is at least, at most, or about 70%, 75%, 80%, 85%, 90%, 95%, 96%, 97%, 98%, 99%, or 100% identical to the wild-type amino acid sequence. The intracellular domain may comprise an intracellular signaling domain of human CD3C and may further comprise human CD28 intracellular segment. In certain aspects the transmembrane domain is a CD28 transmembrane domain.

In a further aspect, compositions may include a nucleic acid encoding the polypeptide described above. In certain aspects the nucleic acid sequence is optimized for human codon usage.

In still a further aspect, compositions may include cells expressing the polypeptide described herein. The T cell may comprise an expression cassette encoding the CAR polypeptide. The expression cassette can be comprised in a non-viral vector, such as a transposon, or a human transposon, or recombinant variant thereof. The expression cassette can be comprised in a viral vector or recombinant variant thereof. The expression cassette can be genomically integrated or episomally maintained or expressed from mRNA.

In yet a further aspect, the disclosure includes a method of making a T cell expressing a human CAR comprising introducing an expression cassette into the cell, wherein the expression cassette encodes a polypeptide comprising an extracellular binding domain, a transmembrane domain, and one or more intracellular signaling domain(s). The method may further comprise stimulating the cells with target antigen, or an antibody to the receptor to cause the cells to proliferate, kill, and/or make cytokines; for example, the cells may be stimulated to proliferate or expand with target antigen-bearing artificial antigen presenting cells.

In certain aspects, the disclosure includes methods of treating a human disease condition comprising infusing a patient with an amount of a recombinant cell expressing a human CAR sufficient to treat the condition, wherein the human CAR comprises an extracellular target binding domain, a transmembrane domain, and an intracellular signaling domain. The condition can be cancer, autoimmune disease or infectious disease, for example.

The hCAR may be a chimeric receptor comprising one or more activation endodomain(s), such as a CD3-ζ-derived activation domain. Additional T-cell activation motifs include, but are not limited to, CD28, CD27, OX-40, DAP10, and 4-1BB. In certain aspects the activation domain can also include a CD28 transmembrane and/or activation domain. In a further aspect the hCAR encoding region and/or expression cassette codon optimized for expression in human cells and subjects, e.g., in one embodiment the scFv region obtained from V_{H} and V_{L} sequences of target-specific human antibodies are incorporated into the binding segment of the hCAR. In another embodiment, the hCAR expression cassette is episomally maintained or integrated into the genome of the recombinant cell. In certain aspects the expression cassette is comprised in a nucleic acid capable of integration by using an integrase mechanism, a viral vector, such as a retroviral vector, or a nonviral vector, such as a transposon mechanism. In a further embodiment the expression cassette is included in a transposon based nucleic acid. In a particular embodiment, the expression cassette is part of a two component Sleeping Beauty (SB) or piggyBac system that utilizes a transposon and transposase for enhanced non-viral gene transfer.

Recombinant hCAR expressing cells can be numerically expanded to clinically-meaningful numbers. One example of such expansion uses artificial antigen presenting cells (aAPC). Recombinant hCAR expressing cells can be verified and identified by flow cytometry and western blot analyses. Recombinant hCAR expressing T cells, expressing a CAR can recognize and kill target cells. In a further aspect, hCAR can be expressed into Universal cells that can be infused across transplantation barriers to help prevent immunogenicity. The hCAR can be used along with human genes for imaging (such as by positron emission tomography, PET) and conditional ablation of T cells, in the event of cytotoxicity. The recombinant cells of the disclosure can be used in specific cellular therapies.

### V. EXEMPLARY MEMBRANE-BOUND IL-15 CO-EXPRESSING CHIMERIC ANTIGEN RECEPTOR OR TRANSGENIC TCR T CELLS FOR TARGETING MINIMALLY RESIDUAL DISEASE

Chemotherapeutic treatment of adult and pediatric B-lineage acute lymphoblastic leukemia (B-ALL) have disease relapse rates of 65% and 20%, respectively, due to drug-resistant residual disease. The high incidence of B-ALL relapse, especially in poor prognostic groups, has prompted the use of immune-based therapies using allogeneic hematopoietic stem cell transplantation (HSCT). This therapy is dependent on alloreactive cells present in the donor graft for the eradication of remaining leukemic cells, or minimal residual disease, to improve disease-free survival. Donor lymphocyte infusions have been used to enhance the ability of engrafted T cells to target residual B-ALL after allogeneic HSCT, but this treatment approach for such patients achieves less than a 10% remission rate and is associated with a high degree of morbidity and mortality from the frequency and severity of graft-versus-host disease (GVHD). With relapse a common and lethal problem in these refractory malignancies, adoptive therapy using peripheral blood mononuclear cells (PBMC)-derived T cells after HSCT may be used to increase the anti-tumor effect, or graft-versus-leukemia (GVL) effect, by retargeting the specificity of donor T cells to a tumor-associated antigen (TAA).

Currently, CAR-modified T cells are reliant on obtaining survival signaling through the CAR which occurs only upon encounter with the tumor antigen. In clinical situations where these CAR-modified T cells are infused into patients with bulky disease, there is ample tumor antigen present to provide sufficient activation and survival signaling via the CAR. However, patients with relapsed B-ALL are often conditioned with myeloablative chemotherapy followed by HSCT and present with minimal residual disease (MRD). In this case, patients have a low tumor load and the minute level of TAA severely restricts the CAR-mediated signaling necessary for supporting the infused T cells consequently compromising therapeutic potential. An alternate CAR-independent means for improving T cell persistence would be anticipated to improve the engraftment of CAR-modified T cells.

Cytokines in the common gamma chain receptor family (γC) are important costimulatory molecules for T cells that are critical to lymphoid function, survival, and proliferation. IL-15 possesses several attributes that are desirable for adoptive therapy. IL-15 is a homeostatic cytokine that supports the survival of long-lived memory cytotoxic T cells, promotes the eradication of established tumors via alleviating functional suppression of tumor-resident cells, and inhibits AICD.

IL-15 is tissue restricted and only under pathologic conditions is it observed at any level in the serum, or systemically. Unlike other γC cytokines that are secreted into the surrounding milieu, IL-15 is trans-presented by the producing cell to T cells in the context of IL-15 receptor alpha (IL-15Rα). The unique delivery mechanism of this cytokine to T cells and other responding cells: (i) is highly targeted and localized, (ii) increases the stability and half-life of IL-15, and (iii) yields qualitatively different signaling than is achieved by soluble IL-15.

In one embodiment, the disclosure provides a method of generating chimeric antigen receptor (CAR)-modified T cells or transgenic TCR T cells with long-lived in vivo potential for the purpose of treating, for example, leukemia patients exhibiting minimal residual disease (MRD). In aggregate, this method describes how soluble molecules such as cytokines can be fused to the cell surface to augment therapeutic potential. The core of this method relies on co-modifying CAR T cells or transgenic TCR T cells with a human cytokine mutein of interleukin-15 (IL-15), henceforth referred to as mIL15. The mIL15 fusion protein is comprised of codon-optimized cDNA sequence of IL-15 fused to the full length IL15 receptor a via a flexible serine-glycine linker. This IL-15 mutein was designed in such a fashion so as to: (i) restrict the mIL15 expression to the surface of the CAR′ or transgenic TCR⁺ T cells to limit diffusion of the cytokine to non-target in vivo environments, thereby potentially improving its safety profile as exogenous soluble cytokine administration has led to toxicities; and (ii) present IL-15 in the context of IL-15Ra to mimic physiologically relevant and qualitative signaling as well as stabilization and recycling of the IL-15/IL-15Ra complex for a longer cytokine half-life. T cells expressing mIL15 are capable of continued supportive cytokine signaling, which is critical to their survival post-infusion. The mIL15⁺CAR⁺ T cells or mIL15⁺transgenic TCR⁺ T cells generated by non-viral Sleeping Beauty System genetic modification and subsequent ex vivo expansion on a clinically applicable platform yielded a T cell infusion product with enhanced persistence after infusion in murine models with high, low, or no tumor burden. Moreover, the mIL15⁺CAR⁺ T cells also demonstrated improved anti-tumor efficacy in both the high or low tumor burden models.

The improved persistence and anti-tumor activity of mIL15⁺CAR⁺ T cells over CAR⁺ T cells in the high tumor burden model indicates that mIL15⁺CAR⁺ T cells may be more efficacious than CAR′ T cells in treating leukemia patients with active disease where tumor burden is prevalent. Thus, mIL15⁺CAR⁺ T cells could supplant CAR′ T cells in adoptive therapy in the broadest of applications. The capability of mIL15⁺CAR⁺ T cells to survive independent of survival signaling via the CAR enables these modified T cells to persist post-infusion despite the lack of tumor antigen. Consequently, this is anticipated to generate the greatest impact in therapeutic efficacy in a MRD treatment setting, especially in patients who have had myeloablative chemotherapy and hematopoietic stem cell transplantation. These patients would receive adoptive T cell transfer with mIL15⁺CAR⁺ T cells to treat their MRD and prevent relapse.

Membrane-bound cytokines, such as mIL15, have broad implications. In addition to membrane-bound IL-15, other membrane-bound cytokines are envisioned. The membrane-bound cytokines can also be extended to cell surface expression of other molecules associated with activating and propagating cells used for human application. These include, but are not limited to cytokines, chemokines, and other molecules that contribute to the activation and proliferation of cells used for human application.

Membrane-bound cytokine, such as mIL15 can be used ex vivo to prepare cells for human application(s) and can be on infused cells (e.g., T cells) used for human application. For example, membrane-bound IL-15 can be expressed on artificial antigen presenting cells (aAPC), such as derived from K562, to stimulate T cells and NK cells (as well as other cells) for activation and/or proliferation. The population of T cells activated/propagated by mIL15 on aAPC includes genetically modified lymphocytes, but also tumor-infiltrating lymphocytes, and other immune cells. These aAPC are not infused. In contrast, mIL15 (and other membrane-bound molecules) can be expressed on T cells, and other cells, that are infused.

Therapeutic efficacy of MRD treatment with CAR-modified T cells is hampered by a lack of persistence after adoptive transfer of the T cells. The capability of mIL15⁺CAR⁺ T cells or mIL15⁺transgenic TCR⁺ T cells to survive long-term in vivo independently of tumor antigen indicates great potential for treating patients with MRD. In this case, mIL15 and the persisting T cells that it supports would address a need, as current approaches for MRD patients are insufficient. The persistence of T cells and other lymphocytes that are infused in patients with MRD applies beyond CAR′ T cells. Any immune cell that is used to treat and prevent malignancy, infection or auto-immune disease must be able to persist over the long term if continued therapeutic impact is to be achieved. Thus, activating T cells for persistence beyond the signal derived from endogenous T-cell receptor or an introduced immunoreceptor is important to many aspects of adoptive immunotherapy. The expression of membrane-bound cytokine(s) thus can be used to augment the therapeutic potential and persistence of T cells and other immune cells infused for a variety of pathologic conditions.

The inventors have generated a mutein of IL-15 that is expressed as a membrane-bound fusion protein of IL-15 and IL-15Rα (mIL15) on CAR⁺ T cells or transgenic TCR⁺ T cells. The mIL15 construct was co-electro-transferred with a CD19-specific CAR (on Day 0) into primary human T cells as two Sleeping Beauty DNA transposon plasmids. Clinically relevant numbers of mIL15⁺CAR⁺ T cells were generated by co-culture on CD19⁺ artificial antigen presenting cells and supplemented IL-21. Signaling through the IL-15 receptor complex in genetically modified T cells was validated by phosphorylation of STATS (pSTAT5) and these T cells demonstrated redirected specific lysis of CD19⁺ tumor targets equivalent to CAR⁺ T cells. Furthermore, after antigen withdrawal, signaling generated by mIL15 increased the prevalence of T cells with a less differentiated/younger phenotype that possessed memory-associated attributes including specific cell surface markers, transcription factors, and the capacity to secrete IL-2. These characteristics are desirable traits in T cells used in adoptive transfer as they are correlated with T cell subsets with demonstrated capability to persist long-term in vivo. In immunocompromised NSG mice bearing a disseminated CD19⁺ leukemia, the mIL15⁺CAR⁺ T cells demonstrated both persistence and an anti-tumor effect whereas its CAR⁺ T cell counterpart could not maintain significant persistence despite the presence of TAA. In a preventative mouse (NSG) model where mIL15^{+/-}CAR⁺ T cells were first engrafted for six days followed by the introduction of a disseminated CD19⁺ leukemia, only the mIL15⁺CAR⁺ T cells were found to persist as well as prevent tumor engraftment. To test whether mIL15⁺CAR⁺ T cells were capable of persistence independent of stimulation from TAA, mIL15^{+/-}CAR⁺ T cells were adoptively transferred into NSG mice with no tumor. Only mIL15⁺CAR⁺ T cells were capable of persisting in this in vivo environment without exogenous cytokine support or the presence of CD19 TAA. These data demonstrate that mIL15 can be co-expressed on CAR′ T cells or transgenic TCR⁺ T cells resulting in enhanced in vivo persistence without the need for TAA or exogenous cytokine support. In summary, this cytokine fusion molecule: (i) provides stimulatory signals via pSTAT5 leading to augmented in vivo T-cell persistence while maintaining tumor-specific functionality, (ii) maintains T-cell subsets that promotes a memory-like phenotype, (iii) eliminates the need and cost for clinical-grade IL-2 for in vitro and in vivo T-cell expansion and persistence, and (iv) mitigates the need for clinical-grade soluble IL-15.

### VI. PANCREATIC ADENOCARCINOMA

Pancreatic cancer arises when cells in the pancreas, a glandular organ behind the stomach, begin to multiply out of control and form a mass. These cancerous cells have the ability to invade other parts of the body. There are a number of types of pancreatic cancer. The most common, pancreatic adenocarcinoma, accounts for about 85% of cases, and the term “pancreatic cancer” is sometimes used to refer only to that type. These adenocarcinomas start within the part of the pancreas which makes digestive enzymes. Several other types of cancer, which collectively represent the majority of the non-adenocarcinomas, can also arise from these cells. One to two percent of cases of pancreatic cancer are neuroendocrine tumors, which arise from the hormone-producing cells of the pancreas. These are generally less aggressive than pancreatic adenocarcinoma.

Signs and symptoms of the most-common form of pancreatic cancer may include yellow skin, abdominal or back pain, unexplained weight loss, light-colored stools, dark urine, and loss of appetite. There are usually no symptoms in the disease's early stages, and symptoms that are specific enough to suggest pancreatic cancer typically do not develop until the disease has reached an advanced stage. By the time of diagnosis, pancreatic cancer has often spread to other parts of the body.

Pancreatic cancer rarely occurs before the age of 40, and more than half of cases of pancreatic adenocarcinoma occur in those over 70. Risk factors for pancreatic cancer include tobacco smoking, obesity, diabetes, and certain rare genetic conditions. About 25% of cases are linked to smoking, and 5-10% are linked to inherited genes. Pancreatic cancer is usually diagnosed by a combination of medical imaging techniques such as ultrasound or computed tomography, blood tests, and examination of tissue samples (biopsy). The disease is divided into stages, from early (stage I) to late (stage IV). Screening the general population has not been found to be effective.

The risk of developing pancreatic cancer is lower among non-smokers, and people who maintain a healthy weight and limit their consumption of red or processed meat. A smoker's chance of developing the disease decreases if they stop smoking and almost returns to that of the rest of the population after 20 years. Pancreatic cancer can be treated with surgery, radiotherapy, chemotherapy, palliative care, or a combination of these. Treatment options are partly based on the cancer stage. Surgery is the only treatment that can cure pancreatic adenocarcinoma and may also be done to improve quality of life without the potential for cure. Pain management and medications to improve digestion are sometimes needed. Early palliative care is recommended even for those receiving treatment that aims for a cure.

In 2015, pancreatic cancers of all types resulted in 411,600 deaths globally. Pancreatic cancer is the fifth most-common cause of death from cancer in the United Kingdom, and the third most-common in the United States. The disease occurs most often in the developed world, where about 70% of the new cases in 2012 originated. Pancreatic adenocarcinoma typically has a very poor prognosis: after diagnosis, 25% of people survive one year and 5% live for five years. For cancers diagnosed early, the five-year survival rate rises to about 20%. Neuroendocrine cancers have better outcomes; at five years from diagnosis, 65% of those diagnosed are living, though survival varies considerably depending on the type of tumor.

### VII. IMMUNE SYSTEM AND IMMUNOTHERAPY

In some embodiments, a medical disorder is treated by transfer of a redirected T cell that elicits a specific immune response. In one embodiment of the present disclosure, B-cell lineage malignancy or disorder is treated by transfer of a redirected T cell that elicits a specific immune response. Thus, a basic understanding of the immunologic responses is necessary.

The cells of the adaptive immune system are a type of leukocyte, called a lymphocyte. B cells and T cells are the major types of lymphocytes. B cells and T cells are derived from the same pluripotent hematopoietic stem cells and are indistinguishable from one another until after they are activated. B cells play a large role in the humoral immune response, whereas T cells are intimately involved in cell-mediated immune responses. They can be distinguished from other lymphocyte types, such as B cells and NK cells by the presence of a special receptor on their cell surface called the T-cell receptor (TCR). In nearly all other vertebrates, B cells and T cells are produced by stem cells in the bone marrow. T cells travel to and develop in the thymus, from which they derive their name. In humans, approximately 1%-2% of the lymphocyte pool recirculates each hour to optimize the opportunities for antigen-specific lymphocytes to find their specific antigen within the secondary lymphoid tissues.

T lymphocytes arise from hematopoietic stem cells in the bone marrow, and typically migrate to the thymus gland to mature. T cells express a unique antigen binding receptor on their membrane (T-cell receptor), which can only recognize antigen in association with major histocompatibility complex (MHC) molecules on the surface of other cells. There are at least two populations of T cells, known as T helper cells and T cytotoxic cells. T helper cells and T cytotoxic cells are primarily distinguished by their display of the membrane bound glycoproteins CD4 and CD8, respectively. T helper cells secret various lymphokines that are crucial for the activation of B cells, T cytotoxic cells, macrophages, and other cells of the immune system. In contrast, T cytotoxic cells that recognize an antigen-MHC complex proliferate and differentiate into effector cell called cytotoxic T lymphocytes (CTLs). CTLs eliminate cells of the body displaying antigen, such as virus infected cells and tumor cells, by producing substances that result in cell lysis. Natural killer cells (or NK cells) are a type of cytotoxic lymphocyte that constitutes a major component of the innate immune system. NK cells play a major role in the rejection of tumors and cells infected by viruses. The cells kill by releasing small cytoplasmic granules of proteins called perforin and granzyme that cause the target cell to die by apoptosis.

Antigen-presenting cells, which include macrophages, B lymphocytes, and dendritic cells, are distinguished by their expression of a particular MHC molecule. APCs internalize antigen and re-express a part of that antigen, together with the MHC molecule on their outer cell membrane. The major histocompatibility complex (MHC) is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex.

The T-cell receptor, or TCR, is a molecule found on the surface of T lymphocytes (or T cells) that is generally responsible for recognizing antigens bound to major histocompatibility complex (MHC) molecules. It is a heterodimer consisting of an α and β chain in 95% of T cells, while 5% of T cells have TCRs consisting of gamma and delta chains. Engagement of the TCR with antigen and MHC results in activation of its T lymphocyte through a series of biochemical events mediated by associated enzymes, co-receptors, and specialized accessory molecules. In immunology, the CD3 antigen (CD stands for cluster of differentiation) is a protein complex composed of four distinct chains (CD3γ, CD3δ, and two times CD3ε) in mammals, that associate with molecules known as the T-cell receptor (TCR) and the ζ-chain to generate an activation signal in T lymphocytes. The TCR, ζ-chain, and CD3 molecules together comprise the TCR complex. The CD3γ, CD3δ, and CD3c chains are highly related cell surface proteins of the immunoglobulin superfamily containing a single extracellular immunoglobulin domain. The transmembrane region of the CD3 chains is negatively charged, a characteristic that allows these chains to associate with the positively charged TCR chains (TCRα and TCRβ). The intracellular tails of the CD3 molecules contain a single conserved motif known as an immunoreceptor tyrosine-based activation motif or ITAM for short, which is essential for the signaling capacity of the TCR.

CD28 is one of the molecules expressed on T cells that provide co-stimulatory signals, which are required for T cell activation. CD28 is the receptor for B7.1 (CD80) and B7.2 (CD86). When activated by Toll-like receptor ligands, the B7.1 expression is upregulated in antigen presenting cells (APCs). The B7.2 expression on antigen presenting cells is constitutive. CD28 is the only B7 receptor constitutively expressed on naive T cells. Stimulation through CD28 in addition to the TCR can provide a potent co-stimulatory signal to T cells for the production of various interleukins (IL-2 and IL-6 in particular).

The strategy of isolating and expanding antigen-specific T cells as a therapeutic intervention for human disease has been validated in clinical trials (Riddell et al., 1992; Walter et al., 1995; Heslop et al., 1996).

Autoimmune disease, or autoimmunity, is the failure of an organism to recognize its own constituent parts (down to the sub-molecular levels) as “self,” which results in an immune response against its own cells and tissues. Any disease that results from such an aberrant immune response is termed an autoimmune disease. Prominent examples include Coeliac disease, diabetes mellitus type 1 (IDDM), systemic lupus erythematosus (SLE), Sjögren's syndrome, multiple sclerosis (MS), Hashimoto's thyroiditis, Graves' disease, idiopathic thrombocytopenic purpura, and rheumatoid arthritis (RA).

Inflammatory diseases, including autoimmune diseases are also a class of diseases associated with B-cell disorders. Examples of autoimmune diseases include, but are not limited to, acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, perniciousanemia, rapidly progressive glomerulonephritis, psoriasis, and fibrosing alveolitis. The most common treatments are corticosteroids and cytotoxic drugs, which can be very toxic. These drugs also suppress the entire immune system, can result in serious infection, and have adverse effects on the bone marrow, liver, and kidneys. Other therapeutics that has been used to treat Class III autoimmune diseases to date have been directed against T cells and macrophages. There is a need for more effective methods of treating autoimmune diseases, particularly Class III autoimmune diseases.

### VIII. ARTIFICIAL ANTIGEN PRESENTING CELLS

In some cases, aAPCs are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009).

aAPCs are typically incubated with a peptide of an optimal length that allows for direct binding of the peptide to the WIC molecule without additional processing. Alternatively, the cells can express and antigen of interest (i.e., in the case of WIC-independent antigen recognition). In addition to peptide-WIC molecules or antigens of interest, the aAPC systems may also comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD70 and B7.1 (B7.1 was previously known as B7 and also known as CD80), which among other things, bind to CD28 and/or CTLA-4 molecules on the surface of T cells, thereby affecting, for example, T-cell expansion, Th1 differentiation, short-term T-cell survival, and cytokine secretion such as interleukin (IL)-2 (see Kim et al., 2004). Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), that promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001.

Cells selected to become aAPCs, preferably have deficiencies in intracellular antigen-processing, intracellular peptide trafficking, and/or intracellular WIC Class I or Class II molecule-peptide loading, or are poikilothermic (i.e., less sensitive to temperature challenge than mammalian cell lines) or possess both deficiencies and poikilothermic properties. Preferably, cells selected to become aAPCs also lack the ability to express at least one endogenous counterpart (e.g., endogenous MHC Class I or Class II molecule and/or endogenous assisting molecules as described above) to the exogenous MHC Class I or Class II molecule and assisting molecule components that are introduced into the cells. Furthermore, aAPCs preferably retain the deficiencies and poikilothermic properties that were possessed by the cells prior to their modification to generate the aAPCs. Exemplary aAPCs either constitute or are derived from a transporter associated with antigen processing (TAP)-deficient cell line, such as an insect cell line. An exemplary poikilothermic insect cells line is a Drosophila cell line, such as a Schneider 2 cell line (see, e.g. Schneider, 1972). Illustrative methods for the preparation, growth, and culture of Schneider 2 cells, are provided in U.S. Pat. Nos. 6,225,042, 6,355,479, and 6,362,001.

In one embodiment, aAPCs are also subjected to a freeze-thaw cycle. In an exemplary freeze-thaw cycle, the aAPCs may be frozen by contacting a suitable receptacle containing the aAPCs with an appropriate amount of liquid nitrogen, solid carbon dioxide (i.e., dry ice), or similar low-temperature material, such that freezing occurs rapidly. The frozen aAPCs are then thawed, either by removal of the aAPCs from the low-temperature material and exposure to ambient room temperature conditions, or by a facilitated thawing process in which a lukewarm water bath or warm hand is employed to facilitate a shorter thawing time. Additionally, aAPCs may be frozen and stored for an extended period of time prior to thawing. Frozen aAPCs may also be thawed and then lyophilized before further use. Preferably, preservatives that might detrimentally impact the freeze-thaw procedures, such as dimethyl sulfoxide (DMSO), polyethylene glycols (PEGs), and other preservatives, are absent from media containing aAPCs that undergo the freeze-thaw cycle, or are essentially removed, such as by transfer of aAPCs to media that is essentially devoid of such preservatives.

In other preferred embodiments, xenogenic nucleic acid and nucleic acid endogenous to the aAPCs, may be inactivated by crosslinking, so that essentially no cell growth, replication or expression of nucleic acid occurs after the inactivation. In one embodiment, aAPCs are inactivated at a point subsequent to the expression of exogenous MHC and assisting molecules, presentation of such molecules on the surface of the aAPCs, and loading of presented MHC molecules with selected peptide or peptides. Accordingly, such inactivated and selected peptide loaded aAPCs, while rendered essentially incapable of proliferating or replicating, retain selected peptide presentation function. Preferably, the crosslinking also yields aAPCS that are essentially free of contaminating microorganisms, such as bacteria and viruses, without substantially decreasing the antigen-presenting cell function of the aAPCs. Thus crosslinking maintains the important APC functions of aAPCs while helping to alleviate concerns about safety of a cell therapy product developed using the aAPCs. For methods related to crosslinking and aAPCs, see for example, U.S. Patent Application Publication No. 20090017000, which is incorporated herein by reference.

### IX. KITS OF THE DISCLOSURE

Any of the compositions described herein may be comprised in a kit. In some embodiments, allogeneic CAR T-cells are provided in the kit, which also may include reagents suitable for expanding the cells, such as media, aAPCs, growth factors, antibodies (e.g., for sorting or characterizing CAR T-cells) and/or plasmids encoding CARs or transposase.

In a non-limiting example, a chimeric receptor expression construct, one or more reagents to generate a chimeric receptor expression construct, cells for transfection of the expression construct, and/or one or more instruments to obtain allogeneic cells for transfection of the expression construct (such an instrument may be a syringe, pipette, forceps, and/or any such medically approved apparatus).

In some embodiments, an expression construct for eliminating endogenous TCR α/β expression, one or more reagents to generate the construct, and/or CAR⁺ T cells are provided in the kit. In some embodiments, there includes expression constructs that encode zinc finger nuclease(s).

In some aspects, the kit comprises reagents or apparatuses for electroporation of cells.

The kits may comprise one or more suitably aliquoted compositions of the present disclosure or reagents to generate compositions of the disclosure. The components of the kits may be packaged either in aqueous media or in lyophilized form. The container means of the kits may include at least one vial, test tube, flask, bottle, syringe, or other container means, into which a component may be placed, and preferably, suitably aliquoted. Where there is more than one component in the kit, the kit also will generally contain a second, third, or other additional container into which the additional components may be separately placed. However, various combinations of components may be comprised in a vial. The kits of the present disclosure also will typically include a means for containing the chimeric receptor construct and any other reagent containers in close confinement for commercial sale. Such containers may include injection or blow molded plastic containers into which the desired vials are retained, for example.

### X. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Mouse microarray analysis. CD8⁺NK1.1⁺ cells and CD8⁺NK1.1^{neg} cells were isolated from mice that previously received pancreatic tumors and were therapeutically treated with cell based vaccine in conjunction with gemcitabine chemotherapy (Konduri et al., 2016). The isolated cells were activated with PDAC antigen-loaded autologous DC and total RNA was isolated using the RNeasy Mini Kit (Qiagen) according to the manufacturer's instructions. Gene expression profiling of CD8⁺NK1.1⁺ and CD8⁺NK1.1^{neg} cells was performed with the Affymetrix Mouse Transcriptome Array 1.0 Chip (Affymetrix, Santa Clara, Calif., USA) by the Sequencing and Microarray Facility at the University of Texas MD Anderson Cancer Center (Houston, Tex.).

Influenza model. To generate T-cells for adoptive transfer, C57/BL6 mice were challenged with influenza A/HongKong/8/68 (H3N2) Swiss mouse lung-adapted strain of H3N2 influenza A virus graciously provided by Dr. Brian Gilbert as described (Liang et al., 2017). Infection was performed in a 20-min nebulized aerosol exposure of influenza virus diluted in MEM media+0.05% gelatin using the Aerotech II nebulizer owing at 10 L/min of room air generated from an Aridyne 2000 compressor. All mice infected in any given experiment were infected simultaneously in a single exposure chamber. Two-weeks post infection, mice were sacrificed, spleens harvested and CD8⁺ cells negatively selected (Miltenyi Biotec). Isolated CD8⁺ cells were further magnetically sorted into NK1.1⁺ and NK1.1^{neg} populations (Miltenyi Biotec). 500,000 CD8⁺NK1.1⁺ and CD8⁺NK1.1^{neg} cells were subsequently adoptively transferred to naïve mice that were then challenged with influenza virus.

Melanoma model. To generate T-cells for adoptive transfer, C57/BL6 mice were subcutaneously inoculated with 250,000 B16F10 melanoma tumor cells (American Type Culture Collection, Manassas, Va.,) suspended in 100 μl PBS. DC were loaded with melanoma tumor antigens as described (Konduri et al., 2016). One-week post tumor inoculation, 200,000 antigen-loaded DC suspended in 50 μl PBS were injected into the footpad with a booster vaccination given seven days later. Ten days post-boost, vaccinated mice were sacrificed, and the CD8⁺ splenocytes were isolated by negative selection (Miltenyi Biotec). Isolated CD8⁺ cells were further magnetically sorted into NK1.1⁺ and NK1.1^{neg} populations (Miltenyi Biotec). Three groups of 8 naïve mice each were given subcutaneous injection of 250,000 B16F10 tumor cells. Tumor sizes were recorded and animals were randomized so that each group possessed similar average tumor sizes and standard errors. Seven days post tumor inoculation, mice in the treatment groups received 1.5 million each of CD8⁺NK1.1⁺ cells or CD8⁺NK1.1⁻ cells by intraperitoneal adoptive transfer. Naïve mice served as untreated controls. Tumor size was determined by external caliper measurement and calculated by means of the formula (length×width²)×π/6. The mice were euthanized 22 days post tumor inoculation once the tumor burden in the control group exceeded the permissible limits set by the Center for Comparative Medicine (CCM).

Analysis of murine PBMC. Two weeks post influenza infection or three weeks post tumor implantation, PBMC were collected from the mice that received adoptive transfer by retro-orbital bleed. Red blood cells were lysed by treatment with ammonium chloride (Sigma-Aldrich) according to the manufacturer's instructions. The white blood cell pellet was washed once with PBS and re-suspended in AIM-V medium with 10% mouse serum. The cells were stained with anti-CD3, CD4, CD8, CD25, IFN-γ, for analysis by flow cytometry. All flow cytometric analysis was performed using an LSR II flow cytometer (BD Biosciences) and analyzed with FlowJo version 10.0.00003 (Tree Star Inc., Ashland, Oreg.) for OS-X.

Human microarray analysis. CD8⁺CD161⁺ and CD8⁺CD161^{neg} cells were magnetically separated from peripheral blood derived from 3 healthy donors and 3 PDAC patients. The isolated cells were not activated. Total RNA from the cells was isolated by RNeasy Mini Kit (Qiagen) according to the manufacturer's instructions. Gene expression profiling of CD8⁺CD161⁺, and CD8⁺CD161^{neg} cells was performed with Affymetrix Human Transcriptome Array 1.0 Chip (Affymetrix, Santa Clara, Calif., USA) by the Sequencing and Microarray Facility, at the University of Texas MD Anderson Cancer Center (Houston, Tex.). A detailed description of sample requirements and data pre-analysis is available on the facility's web site (world-wide-web at mdanderson.org/research/research-resources/core-facilities/sequencing-and-microarray-facility-smf/services-and-fees/microarray-services-overview.html). Data were analyzed and visualized with Transcriptome Analysis Console v3.0 (Affymetrix).

TCR Vβ spectratyping. CD8⁺CD161⁺ cells isolated from the peripheral blood of a normal donor were spectratyped by the Mayo Clinic. The resulting images are a cluster of fluorescent peaks with single-base-pair separation and different fluorescent intensities, approximately corresponding to the number of fragments of that size represented in the donor's original RNA. The peak patterns were reviewed for organization (number of peaks), relative intensity across peaks, and size distribution.

Cytotoxicity assay. To assess the cytotoxic capacity of CD8⁺CD161⁺ cells against that of CD8⁺CD161^{neg} cells and bulk PBMC, a chromium-based short-term cytotoxicity assay was performed in vitro. CD8⁺CD161⁺, CD8⁺CD161^{neg}, and unmanipulated bulk PBMC were freshly isolated from a human peripheral blood product. Isolated cells were immediately tested for cytotoxic capacity in a four-hour killing assay using ⁵¹Cr-labeled allogeneic 293-HEK targets at 5:1, 25:1 and 50:1 T-cell:target cell ratios. Cell lysis was determined by chromium released into the media, read using a Wizard2 gamma counter (Perkin Elmer).

CD8⁺CD161⁺ cell ex vivo culture conditions. CD8⁺CD161⁺ cells, CD8⁺CD161^{neg} cells, and bulk PBMC isolated from normal donor apheresis products were stimulated with plate bound anti-CD3/CD28 and expanded in a cytokine cocktail comprised of 10 ng/ml IL-7, 5 ng/ml, IL-15, and 30 ng/ml, and IL-21 (all from Peprotech, Rocky Hill, N.J.). CD8⁺CD161⁺ cells were isolated from healthy donor apheresis product and cultured against stimulation with anti-CD3/CD28/Clec2d (anti-human CD3-eBioscience cat #16-0037-8, anti-human CD28 from BD Biosciences cat #555725, recombinant human Clec2d, Novus Biologicals cat #NBP2-22966) each at 1 ug/mL and expanded in RPMI-1640, 10% FBS and 2 mmol/1 GlutaMAX (Invitrogen) in a cytokine cocktail comprised of 10 ng/ml IL-7, 5 ng/ml, IL-15, and 30 ng/ml, IL-21 (all from Peprotech, Rocky Hill, N.J.). The cells were placed in a humified chamber at 37° C. for 48 hours. After 48 hours, the cells were expanded with the IL7/15/21 cytokine cocktails without antibody stimulation.

Statistical Analysis. Significance of differences was determined by two-way analysis of variance (ANOVA) or one-way ANOVA using the Bonferroni post hoc test for multiple comparisons unless indicated otherwise. Kaplan-Meier survival significance was determined by the log rank (Mantel-Cox) test. All data are displayed as the mean±SEM, and all analyses were performed using Prism software (GraphPad Software) unless indicated otherwise. Statistical significance was defined as p≤0.05.

### Example 2—Results

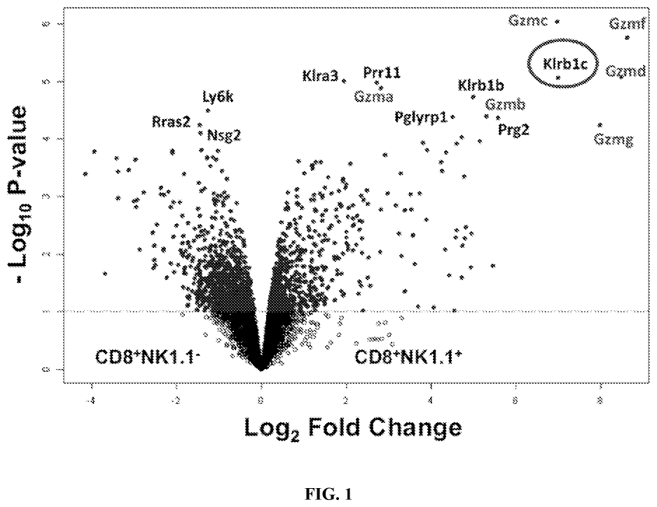

T-cell expression profiling following chemo-immunotherapy of PDAC identifies innate-like and cytotoxic characteristics of CD3⁺CD8⁺NK1.1⁺ cells. Previous work demonstrated that very small numbers (<1,500 per mouse) of splenic CD8⁺NK1.1⁺ cells isolated nine months after chemo-immunotherapy for the treatment of orthotopic PDAC could still provide rapid and robust anti-tumor protection against the parent PDAC cell line in a model of metastatic disease (Konduri et al., 2016). To gain insight into key functional characteristics of this NK1.1⁺CD3⁺CD8⁺ T-cell subset, the inventors orthotopically implanted cohorts of mice with Kras^{G12D}/p53^{-/-} PDAC tumors and subsequently cured them by means of the previously published (Konduri et al., 2016) immune-based therapeutic protocol. Two months after treatment and cure, CD8⁺ splenocytes were negatively selected and subdivided into NK1.1⁺ and NK1.1^{neg} fractions. These fractions were then separately co-cultured overnight with PDAC-loaded matured DC, and the PDAC antigen-specific cells were identified and isolated by upregulated CD69 expression. Microarray indicated that 1642 genes were differentially regulated between CD8⁺NK1.1⁺ and CD8⁺NK1.1^{neg} cells upon antigenic stimulation at a univariate significance level of 0.1 (FIG. 1). While many different pathways were potentially impacted (Table 1), the most striking differences were found in the cytolytic granzyme serine proteases, particularly noncanonical granzyme isoforms F, D, G, and C, as well as among the innate-like cytotoxicity receptors (Table 2). These results suggested CD8⁺NK1.1⁺ cells represent a population of CD8⁺ T cells with significantly enhanced cytolytic capacity.

TABLE 1
Top upregulated and downregulated genes.

| | | | | | |
|---|---|---|---|---|---|
| Gene | NK1.1⁺ | NK1.1^{neg} | Fold | | |
| Symbol | Avg (log2) | Avg (log2) | Change | P | Description |

Top 25 upregulated genes

| | | | | | |
|---|---|---|---|---|---|
| Gzmf | 14.8 | 6.2 | 397.0 | 0.00001 | granzyme F |
| Gzmd | 13.8 | 5.4 | 341.3 | 0.00006 | granzyme D |
| Gzmg | 13.8 | 5.9 | 231.2 | 0.0003 | granzyme G |
| Gzmc | 18.5 | 11.6 | 126.4 | 0.000008 | granzyme C |
| Klrb1c | 11.9 | 5.0 | 125.2 | 0.00006 | killer cell lectin-like |
| | | | | | receptor subfamily B |
| | | | | | member 1C |
| Cd244 | 10.8 | 4.9 | 58.5 | 0.002 | CD244 natural killer cell |
| | | | | | receptor 2B4 |
| Prg2 | 14.3 | 8.6 | 50.8 | 0.0002 | proteoglycan 2, bone |
| | | | | | marrow |
| Igkv4-63 | 12.6 | 7.2 | 43.9 | 0.03 | immunoglobulin kappa |
| | | | | | variable 4-63 |
| Ear12 | 10.4 | 5.1 | 40.0 | 0.002 | eosinophil-associated, |
| | | | | | ribonuclease A family, |
| | | | | | member 12 |
| Igkv5-39 | 9.8 | 4.5 | 39.8 | 0.01 | immunoglobulin kappa |
| | | | | | variable 5-39 |
| Trgj1 | 15.0 | 9.7 | 39.7 | 0.0002 | T cell receptor gamma |
| | | | | | joining 1 |
| Ighv3-6 | 9.7 | 4.5 | 38.6 | 0.009 | immunoglobulin heavy |
| | | | | | variable 3-6 |
| Gzmb | 17.5 | 12.3 | 37.8 | 0.0002 | granzyme B |
| Tcrg-C3 | 15.9 | 10.8 | 35.9 | 0.0005 | T cell receptor gamma, |
| | | | | | constant 3 |
| Klrb1b | 10.5 | 5.5 | 31.6 | 0.0001 | killer cell lectin-like |
| | | | | | receptor subfamily B |
| | | | | | member 1B |
| Igkv4-54 | 11.2 | 6.2 | 31.6 | 0.01 | immunoglobulin kappa |
| | | | | | chain variable 4-54 |
| Igkv4-55 | 10.5 | 5.6 | 30.9 | 0.03 | immunoglobulin kappa |
| | | | | | variable 4-55 |
| Cpa3 | 9.8 | 4.9 | 29.3 | 0.0005 | carboxypeptidase A3, mast |
| | | | | | cell |
| Mcpt8 | 10.7 | 5.9 | 27.9 | 0.008 | mast cell protease 8 |
| Klra7 | 15.0 | 10.3 | 26.5 | 0.0004 | killer cell lectin-like |
| | | | | | receptor, subfamily A, |
| | | | | | member 7 |
| Igkv4-57 | 11.0 | 6.2 | 26.2 | 0.04 | immunoglobulin kappa |
| | | | | | variable 4-57 |
| Igkv4-62 | 10.5 | 5.8 | 26.1 | 0.02 | immunoglobulin kappa |
| | | | | | variable 4-62 |
| Klra6 | 11.7 | 7.0 | 25.9 | 0.001 | killer cell lectin-like |
| | | | | | receptor, subfamily A, |
| | | | | | member 6 |
| Igkv4-59 | 11.5 | 6.9 | 24.8 | 0.01 | immunoglobulin kappa |
| | | | | | variable 4-59 |
| Igkv4-71 | 11.3 | 6.7 | 23.8 | 0.01 | immunoglobulin kappa |
| | | | | | chain variable 4-71 |

Top 25 downregulated genes

| | | | | | |
|---|---|---|---|---|---|
| Slamf6 | 9.2 | 13.5 | −19.8 | 0.001 | SLAM family member 6 |
| Tagap | 10.6 | 14.6 | −16.5 | 0.0007 | T cell activation Rho |
| | | | | | GTPase activating protein |
| Sell | 13.3 | 16.8 | −11.8 | 0.0008 | selectin, lymphocyte |
| Uck2 | 8.0 | 11.6 | −11.7 | 0.04 | uridine-cytidine kinase 2 |
| Gzmk | 7.9 | 11.3 | −10.7 | 0.003 | granzyme K |
| Tcf7 | 10.8 | 14.2 | −10.4 | 0.001 | transcription factor 7, T cell |
| | | | | | specific |
| Dapl1 | 6.7 | 10.0 | −9.8 | 0.0007 | death associated protein- |
| | | | | | like 1 |
| Trbv29 | 8.1 | 11.4 | −9.6 | 0.003 | T cell receptor beta, |
| | | | | | variable 29 |
| Fas | 8.0 | 11.1 | −8.8 | 0.001 | Fas (TNF receptor |
| | | | | | superfamily member 6) |
| Ay036118 | 8.6 | 11.7 | −8.7 | 0.005 | cDNA sequence AY036118 |
| Cd8b1 | 14.2 | 17.2 | −8.1 | 0.0008 | CD8 antigen, beta chain 1 |
| F2rl1 | 5.1 | 8.1 | −7.7 | 0.004 | coagulation factor II |
| | | | | | (thrombin) receptor-like 1 |
| Rs5-8s1 | 14.5 | 17.4 | −7.3 | 0.02 | 5.8S ribosomal RNA |
| Tlr1 | 7.5 | 10.3 | −7.0 | 0.02 | toll-like receptor 1 |
| Trbv20 | 8.1 | 10.9 | −6.8 | 0.003 | T cell receptor beta, |
| | | | | | variable 20 |
| Igfbp4 | 6.9 | 9.6 | −6.6 | 0.0007 | insulin-like growth factor |
| | | | | | binding protein 4 |
| Itgae | 7.0 | 9.8 | −6.6 | 0.003 | integrin alpha E, epithelial- |
| | | | | | associated |
| Cd69 | 13.0 | 15.5 | −5.9 | 0.01 | CD69 antigen |
| Il6st | 8.8 | 11.3 | −5.9 | 0.004 | interleukin 6 signal |
| | | | | | transducer |
| Irf4 | 6.4 | 8.9 | −5.9 | 0.02 | interferon regulatory factor |
| | | | | | 4 |
| Zc3h12d | 8.0 | 10.5 | −5.8 | 0.003 | zinc finger CCCH type |
| | | | | | containing 12D |
| N6amt2 | 8.1 | 10.7 | −5.8 | 0.03 | N-6 adenine-specific DNA |
| | | | | | methyltransferase 2 |
| | | | | | (putative) |
| Bzw2 | 8.5 | 11.1 | −5.7 | 0.02 | basic leucine zipper and W2 |
| | | | | | domains 2 |
| Gbp2 | 10.1 | 12.5 | −5.6 | 0.03 | guanylate binding protein 2 |
| Tagap1 | 8.2 | 10.7 | −5.4 | 0.008 | T cell activation GTPase |
| | | | | | activating protein 1 |

TABLE 2
Fold change and P values for the genes grouped into the granzyme
pathway and killer cell like receptor subfamily pathway

| Gene | NK1.1⁺ | NK1.1^{neg} | Fold | | |
|---|---|---|---|---|---|
| Symbol | Avg (log2) | Avg (log2) | Change | P | Description |
| Gzmf | 14.82 | 6.18 | 397.0 | 0.00001 | granzyme F |
| Gzmd | 13.8 | 5.39 | 341.3 | 0.00006 | granzyme D |
| Gzmg | 13.78 | 5.93 | 231.2 | 0.0003 | granzyme G |
| Gzmc | 18.53 | 11.55 | 126.4 | 0.000008 | granzyme C |
| Gzmb | 17.52 | 12.28 | 37.8 | 0.0002 | granzyme B |
| Gzma | 19.29 | 16.56 | 6.7 | 0.00005 | granzyme A |
| Gzmn | 5.55 | 4.3 | 2.4 | 0.004 | granzyme N |
| Gzmk | 7.85 | 11.27 | −10.7 | 0.003 | granzyme K |
| Klrb1c | 11.93 | 4.97 | 125.2 | 0.00006 | killer cell lectin-like |
| (NK1.1) | | | | | receptor subfamily B |
| | | | | | member 1C |
| Cd244 | 10.79 | 4.92 | 58.5 | 0.002 | CD244 natural killer cell |
| | | | | | receptor 2B4 |
| Klrb1b | 10.52 | 5.54 | 31.6 | 0.0001 | killer cell lectin-like |
| | | | | | receptor subfamily B |
| | | | | | member 1B |
| Klra7 | 15 | 10.27 | 26.5 | 0.0004 | killer cell lectin-like |
| | | | | | receptor, subfamily A, |
| | | | | | member 7 |
| Klra6 | 11.69 | 6.99 | 25.9 | 0.001 | killer cell lectin-like |
| | | | | | receptor, subfamily A, |
| | | | | | member 6 |
| Klrb1a | 8.76 | 4.28 | 22.4 | 0.0007 | killer cell lectin-like |
| | | | | | receptor, subfamily B, |
| | | | | | member 1A |
| Klra5 | 10.17 | 6.37 | 13.9 | 0.0005 | killer cell lectin-like |
| | | | | | receptor, subfamily A, |
| | | | | | member 5 |
| Klre1 | 11.85 | 8.07 | 13.7 | 0.001 | killer cell lectin-like |
| | | | | | receptor family E member 1 |
| Klra9 | 10.53 | 8.05 | 5.6 | 0.004 | killer cell lectin-like |
| | | | | | receptor, subfamily A, |
| | | | | | member 9 |
| Klrk1 | 8.5 | 6.38 | 4.4 | 0.01 | killer cell lectin-like |
| | | | | | receptor, subfamily K, |
| | | | | | member 1 |
| Klra10 | 9.48 | 7.43 | 4.1 | 0.004 | killer cell lectin-like |
| | | | | | receptor, subfamily A, |
| | | | | | member 10 |
| Klra23 | 10.21 | 8.18 | 4.1 | 0.0003 | killer cell lectin-like |
| | | | | | receptor, subfamily A, |
| | | | | | member 23 |
| Klra3 | 9.37 | 7.43 | 3.8 | 0.00002 | killer cell lectin-like |
| | | | | | receptor, subfamily A, |
| | | | | | member 3 |
| Klra16 | 6.93 | 5.09 | 3.6 | 0.004 | killer cell lectin-like |
| | | | | | receptor, subfamily A, |
| | | | | | member 16 |
| Klrc2 | 8.59 | 6.76 | 3.6 | 0.007 | killer cell lectin-like |
| | | | | | receptor, subfamily C, |
| | | | | | member 2 |
| Klra20 | 5.83 | 4.18 | 3.1 | 0.004 | killer cell lectin-like |
| | | | | | receptor, subfamily A, |
| | | | | | member 20 |
| Klrd1 | 15.43 | 13.79 | 3.1 | 0.007 | killer cell lectin-like |
| | | | | | receptor, subfamily D, |
| | | | | | member 1 |
| Klri1 | 6.05 | 4.44 | 3.1 | 0.01 | killer cell lectin-like |
| | | | | | receptor family I member 1 |
| Klrc1 | 9.32 | 7.8 | 2.9 | 0.0006 | killer cell lectin-like |
| | | | | | receptor, subfamily C, |
| | | | | | member 1 |
| Klrc3 | 7.33 | 5.84 | 2.8 | 0.002 | killer cell lectin-like |
| | | | | | receptor, subfamily C, |
| | | | | | member 3 |
| Nkg7 | 15.72 | 14.29 | 2.7 | 0.003 | natural killer cell group 7 |
| | | | | | sequence |
| Klra8 | 6.41 | 5.1 | 2.5 | 0.004 | killer cell lectin-like |
| | | | | | receptor, subfamily A, |
| | | | | | member 8 |
| Klrb1f | 7.68 | 6.57 | 2.2 | 0.02 | killer cell lectin-like |
| | | | | | receptor, subfamily B, |
| | | | | | member 1F |
| Klri2 | 6.32 | 5.28 | 2.1 | 0.008 | killer cell lectin-like |
| | | | | | receptor family I member 2 |
| Klrb1 | 5.65 | 5 | 1.6 | 0.003 | killer cell lectin-like |
| | | | | | receptor, subfamily B, |
| | | | | | member 1 |
| Klrg1 | 5.15 | 4.73 | 1.3 | 0.006 | killer cell lectin-like |
| | | | | | receptor, subfamily G, |
| | | | | | member 1 |
| Klra17 | 4.4 | 4.01 | 1.3 | 0.01 | killer cell lectin-like |
| | | | | | receptor, subfamily A, |
| | | | | | member 17 |

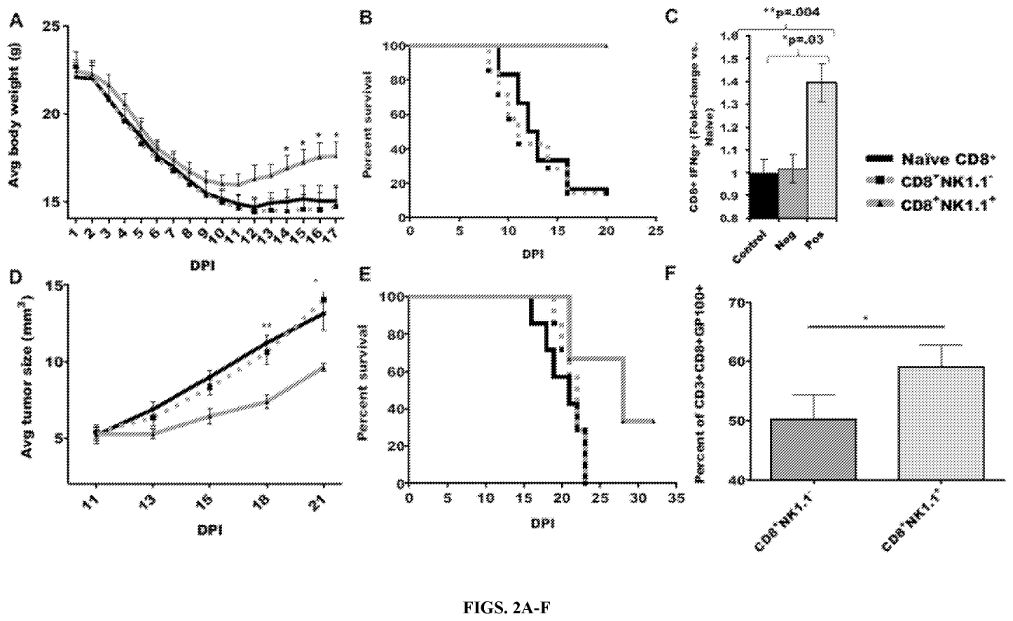

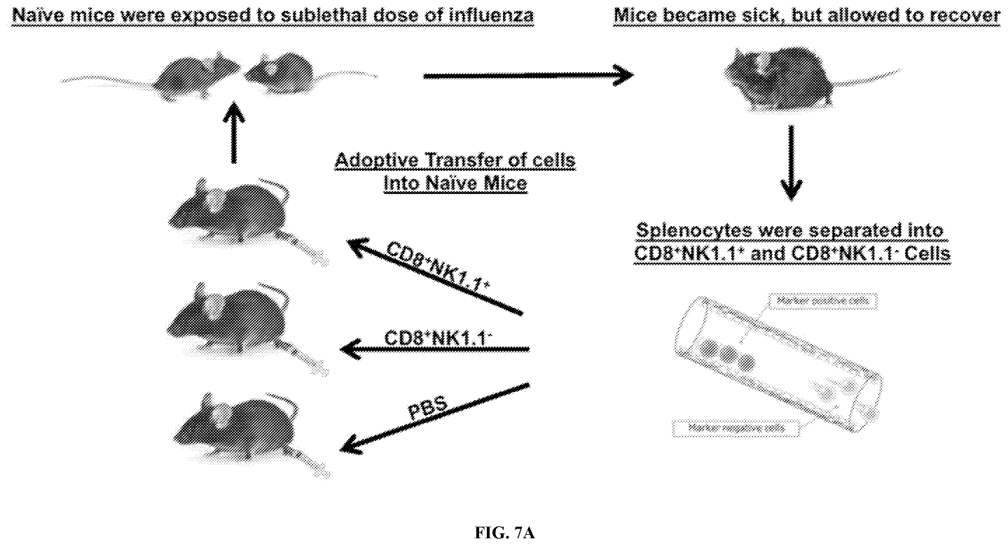

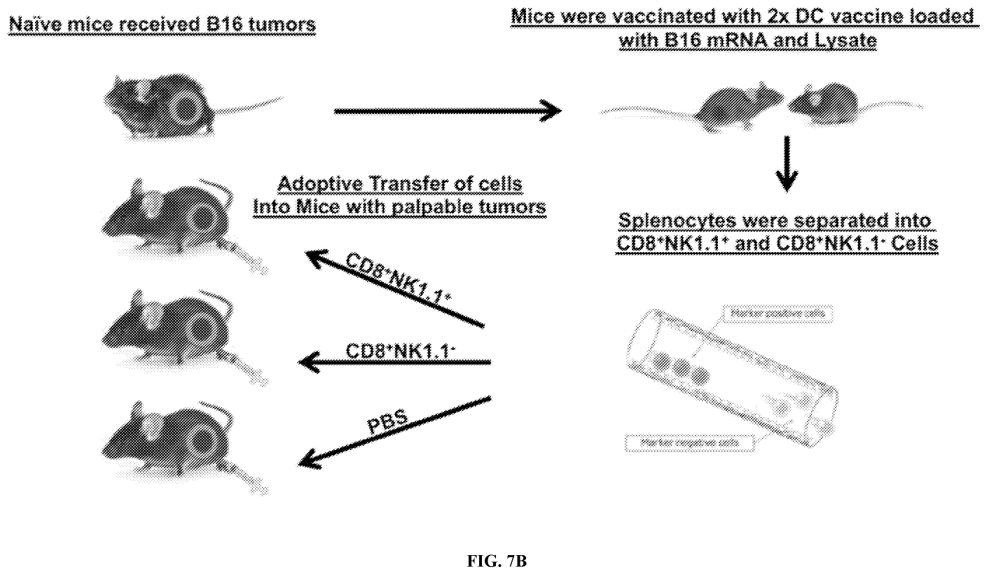

NK1.1 identifies the critical circulating memory T-cell population in multiple mouse models of disease. To validate that NK1.1 could identify a similar critical population of cytolytic memory cells in a model-independent fashion, the inventors performed adoptive transfer experiments in a second tumor model and in an infectious disease model. First, a donor cohort of 6-8 week-old mice was inoculated with a sub-lethal dose of H2N3 mouse-adapted influenza virus. Three weeks after inoculation and recovery from weight loss, splenocytes were harvested and CD8⁺ non-adherent cells isolated by negative selection. After separation into NK1.1⁺ and NK1.1^{neg} fractions by positive selection, 5×10⁵ cells/mouse of each NK1.1 group were adoptively transferred into naïve cohorts that were lethally challenged with the same influenza virus strain 24 hours after adoptive transfer (FIG. 7A). Body weights were recorded as an index of recovery and survival was determined by Kaplan-Meier analysis. The cohort adoptively transferred with donor CD8⁺NK1.1⁺ cells regained full body weight and survived the infection whereas those adoptively transferred with CD8⁺NK1.1^{neg} cells all lost weight and died at the same rate as controls adoptively transferred with naïve CD8⁺ splenocytes (FIGS. 2A-B). Analysis of PBMC at post-infection day 7 showed a 40% increase in circulating CD3⁺CD8⁺IFN-γ⁺ cells (p<0.003) among mice that received CD8⁺NK1.1⁺ cells in comparison to naïve and CD8⁺NK1.1^{neg} adoptively transferred cohorts (FIG. 2C).

In a second model system, a cohort of donor mice was inoculated s.c. with 2×10⁵ B16 melanoma cells and vaccinated with a B16-loaded cell-based vaccine at 7- and 14-days post-inoculation. At day 21, the mice were sacrificed, and splenocytes again harvested and sorted into CD8⁺NK1.1⁺ and CD8⁺NK1.1^{neg} cell populations. Naïve cohorts inoculated with palpable B16 tumors were then each adoptively transferred with 1.5×10⁶ CD8⁺NK1.1⁺ or CD8⁺NK1.1^{neg} cells (FIG. 7B). The mice that received the CD8⁺NK1.1⁺ cells exhibited significant delay in tumor growth accompanied by survival benefit whereas the cohort that received CD8⁺NK1.1^{neg} cells survived identically to the control cohort adoptively transferred with naïve splenocytes (FIGS. 2D-E). Analysis of peripheral blood lymphocytes indicated significantly elevated levels of memory markers CD62L and CCR7 among GP100 tetramer-specific CD8⁺ cells in the cohort adoptively transferred with CD8⁺NK1.1⁺ cells in comparison to cohorts adoptively transferred with CD8⁺NK1.1^{neg} or naïve splenocytes (FIG. 2F). Together these results suggest that the CD161 homolog NK1.1 defines a major CD8^{+ memory cell population in simple models of disease among adolescent mice subjected to a single pathogenic insult.}

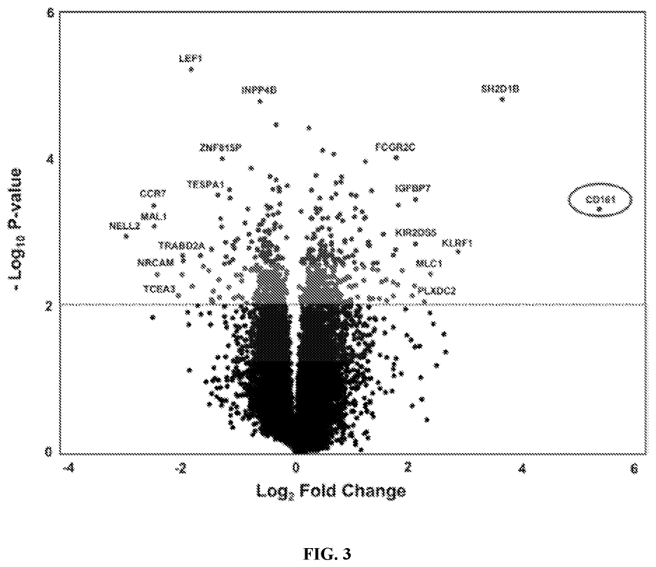

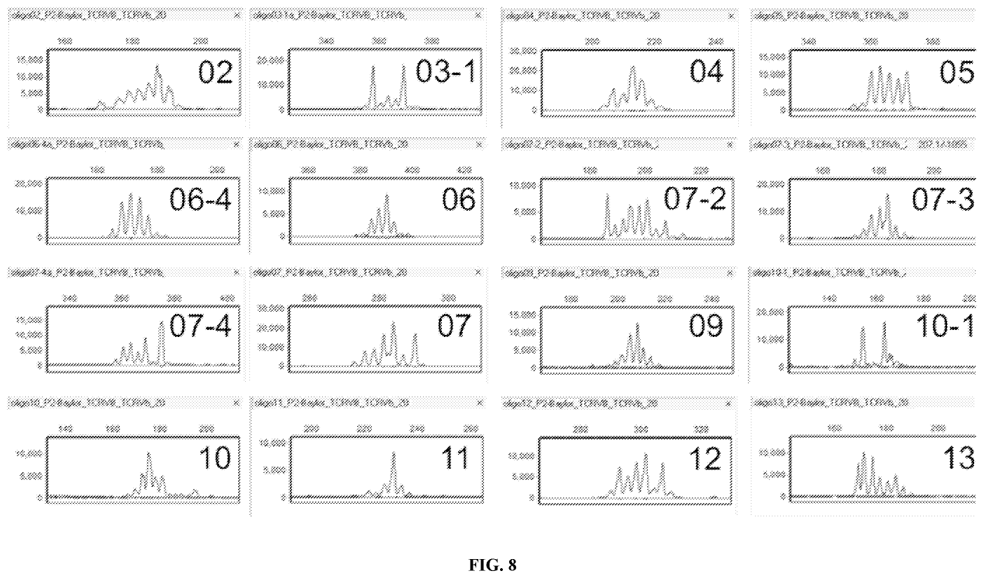

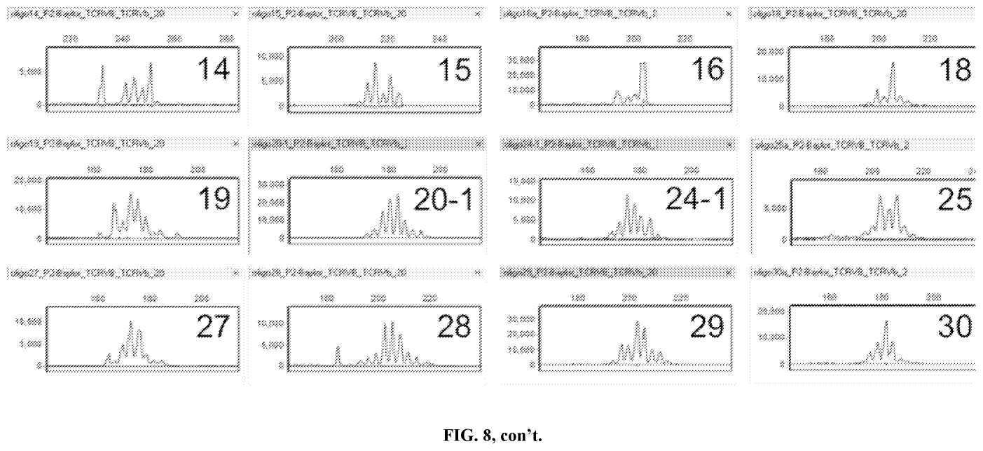

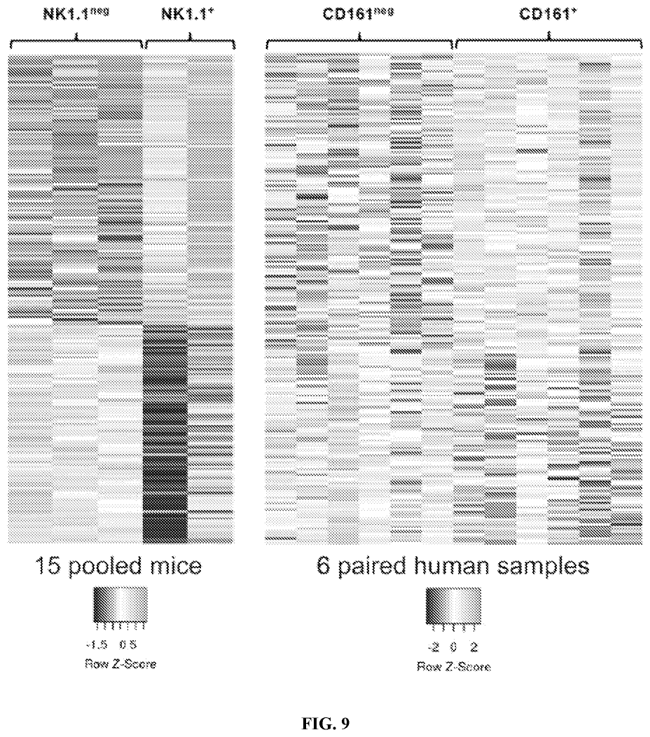

The murine CD3⁺CD8⁺NK1.1⁺ cell population is phenotypically preserved among human CD3⁺CD8⁺CD161⁺ counterparts. Encouraged by the protective memory responses offered by the CD8⁺NK1.1⁺ cells in a variety of systems, the inventors next asked if the subset of memory CD8⁺ T cells defined by NK1.1 expression was phenotypically and transcriptionally conserved in human populations among the analogous CD3⁺CD8⁺CD161⁺ cell population in peripheral circulation. For this analysis CD8⁺CD161⁺ and CD8⁺CD161^{neg} cells were differentially isolated from six different human donors. After verifying that the CD161⁺ cells were polyclonal by TCR-Vβ spectratyping (FIG. 8), transcriptional profiling of each population was performed by microarray analysis. Despite the fact that these cells were not activated prior to analysis and in a homeostatic resting state, the profile of upregulated granzymes and natural cytotoxicity receptors was recapitulated in these cells at a univariate significance level of 0.1 (FIG. 3; Table 3). Cross-species comparative analysis of genes between the activated mouse and unactivated human cells identified a conserved signature of 206 genes with common nomenclatures differentially regulated among the two populations (FIG. 9). Reactome Pathway analysis of the upregulated human genes identified signatures associated with differentiation and regulation at an FDR of <5×10⁻⁴ including HDAC deacetylation, DNA and histone methylation, nucleosome assembly, RNA polymerase I promoter escape, transcriptional regulation by small RNAs, and gene silencing by RNA.

TABLE 3
The phenotypic characteristics of murine CD8⁺NK1.1⁺ cells
are recapitulated in the resting phase of CD8⁺CD161⁺ cells
with elevated expression of granzymes and killer lectin ike receptor genes

| Gene | CD161⁺ | CD161^{neg} | Fold | | |
|---|---|---|---|---|---|
| Symbol | Avg (log2) | Avg (log2) | Change | P | Description |
| GZMB | 7.5 | 6.6 | 1.9 | 0.001 | granzyme B |
| GZMH | 9.6 | 8.9 | 1.6 | 0.001 | granzyme H |
| KLRB1 | 10.3 | 5.5 | 40.9 | 0.0005 | killer cell lectin-like |
| (CD161) | | | | | receptor subfamily B, |
| | | | | | member 1 |
| KLRF1 | 6.7 | 4.1 | 7.3 | 0.002 | killer cell lectin-like |
| | | | | | receptor subfamily F, |
| | | | | | member 1 |
| KIR2DS5 | 5.9 | 4.0 | 4.4 | 0.001 | killer cell immunoglobulin- |
| | | | | | like receptor, two domains, |
| | | | | | short cytoplasmic tail, 5 |
| CD244 | 4.7 | 3.7 | 2.1 | 0.005 | CD244 molecule, natural |
| | | | | | killer cell receptor 2B4 |
| KLRD1 | 8.3 | 7.4 | 2.0 | 0.01 | killer cell lectin-like |
| | | | | | receptor subfamily D, |
| | | | | | member 1 |
| KLRG1 | 8.9 | 8.0 | 2.0 | 0.02 | killer cell lectin-like |
| | | | | | receptor subfamily G, |
| | | | | | member 1 |
| KIR3DX2 | 3.3 | 2.4 | 1.9 | 0.01 | killer cell immunoglobulin- |
| | | | | | like receptor, three |
| | | | | | domains, X1 |
| NKG7 | 11.5 | 11.0 | 1.5 | 0.03 | natural killer cell granule |
| | | | | | protein 7 |
| KLRF2 | 1.4 | 1.7 | −1.2 | 0.03 | killer cell lectin-like |
| | | | | | receptor subfamily F, |
| | | | | | member 2 |

Development of a model system for CAR T-cell therapy of PDAC. Based on the potential of its novel biology, the inventors hypothesized that the human CD8⁺CD161⁺ subset might be able to offer more functional and durable anti-tumor efficacy in the context of solid tumor CAR T-cell therapy than conventional bulk PBMC.

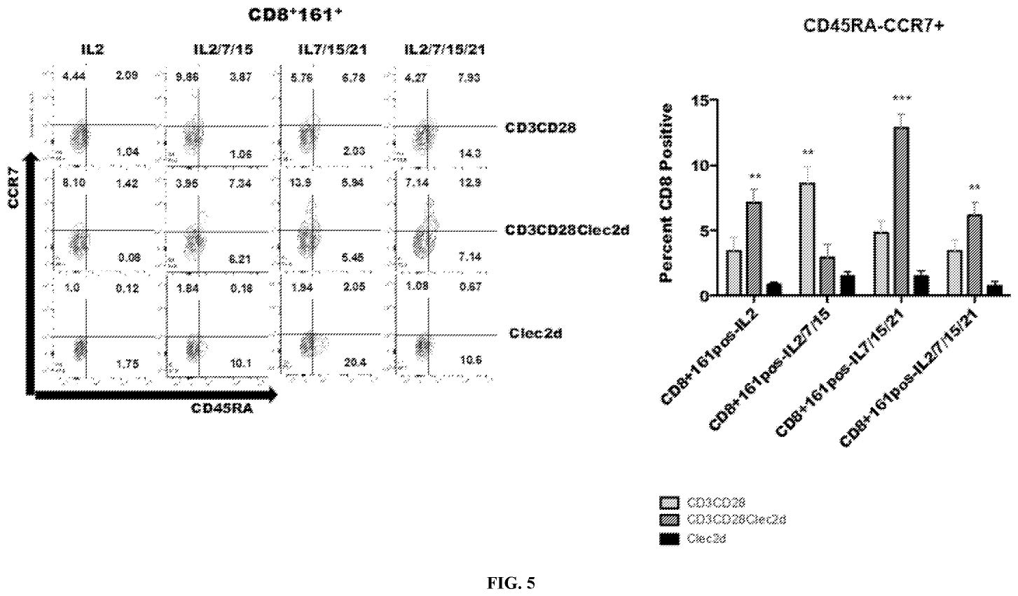

Ex vivo expansion of CD8⁺CD161⁺ cells with IL7/15/21 in combination with plate bound stimulation with anti-CD3/CD28/Clec2d enhanced the central memory phenotype (CD45RA⁻CCR7⁺). CD8⁺CD161⁺ cells were sorted from normal donor and ex vivo stimulation conditions were optimized. Compared to IL2, IL-2/7/15, IL2/7/15/21 stimulation, a combination of IL7/15/21 with plate bound stimulation of anti-CD3/CD28/Clec2d resulted in significant upregulation of central memory (CD45RA⁻CCR7⁺) (FIG. 5).

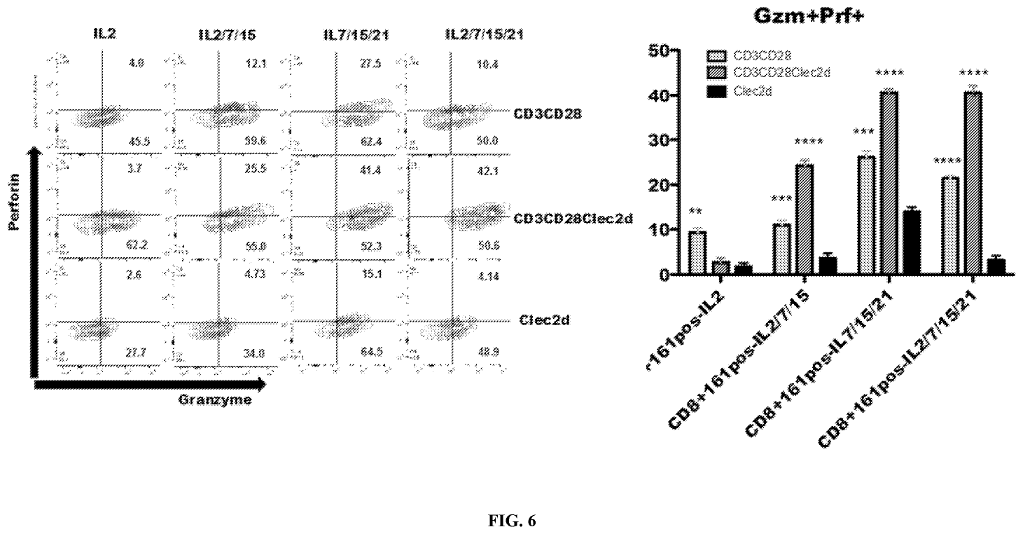

Ex vivo expansion of CD8⁺CD161⁺ cells with IL7/15/21 in combination with plate bound stimulation with anti-CD3/CD28/Clec2d enhanced the cytotoxic granzyme production. CD8⁺CD161⁺ cells were sorted from normal donor and ex vivo stimulation conditions were optimized. Compared to IL2, IL-2/7/15, IL2/7/15/21 stimulation, a combination of IL7/15/21 with plate bound stimulation of anti-CD3/CD28/Clec2d resulted in significant upregulation of cytotoxic molecules, granzyme and perforin (FIG. 6).

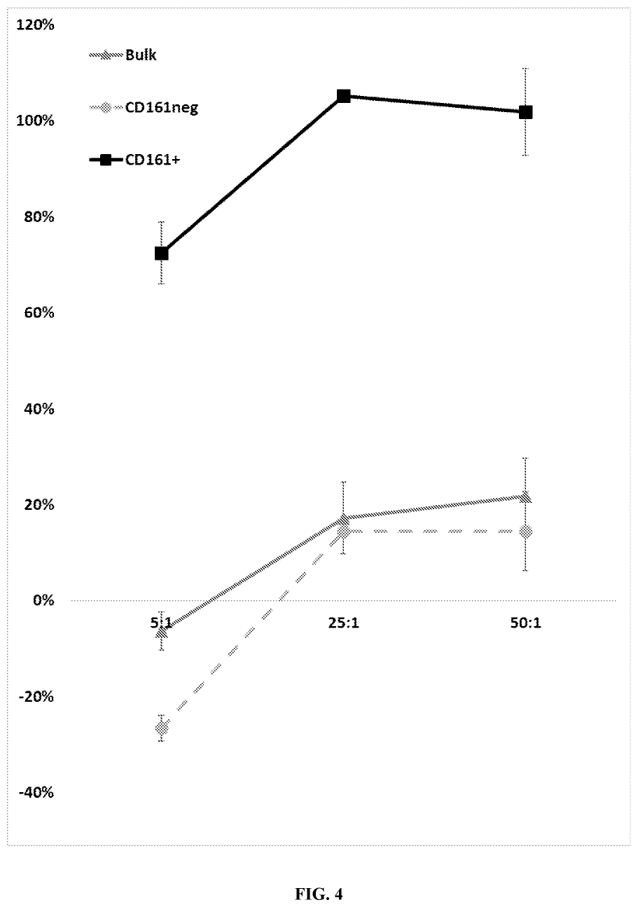

CD8⁺CD161⁺ cells exhibit an inherent killing advantage in vitro. To assess the cytotoxic capacity of CD8⁺CD161⁺ cells against that of CD8⁺CD161^{neg} cells and bulk PBMC, a chromium-based short-term cytotoxicity assay was performed in vitro. CD8⁺CD161⁺, CD8⁺CD161^{neg}, and unmanipulated bulk PBMC were freshly isolated from a human peripheral blood product. Isolated cells were immediately tested for cytotoxic capacity in a four-hour killing assay using ⁵¹Cr-labeled allogeneic 293-HEK targets. As shown in FIG. 4, CD8⁺CD161⁺ cells induced 100% target lysis at an E:T ratio of 25:1 whereas bulk PBMC and CD8⁺CD161^{neg} cells exhibited lytic capabilities of 22% and 15%, respectively, at the maximum E:T ratio of 50:1 (p<0.002 at 50:1, p<0.0007 at 25:1, and p<0.00002 at 5:1 by One-way ANOVA). These data indicated that CD8⁺CD161⁺ T cells have enhanced cytotoxicity not duplicated among CD8⁺CD161^{neg} or bulk PBMC counterparts.

### Example 3—Discussion

Based on the expression of surface molecules and secreted cytokines, lymphocytes are classified into different subsets and lineages. However, the classification is dynamic with the identification of new cell subsets that occasionally express markers from previously identified cell subsets and lineages. One such surface molecule is CD161, known to be expressed on NK cells, NKT cells and other T cell lineages (Fergusson et al., 2011). CD161 shares 47% homology with the murine counterpart NK1.1 and is expressed by up to one quarter of peripheral T-cells (Neelapu et al., 1994). Since NK-T cells comprise less than 1% of peripheral T cells, CD3⁺CD161⁺ cells represent a distinct lineage of T cells given that they comprise more than 5% of circulating T cells (Takahashi et al., 2006). On CD8⁺ T cells, CD161 expression is defined as either intermediate or high while such distinction is not made among CD4⁺ T-cells that express CD161 (Takahashi et al., 2006). CD8⁺CD161^{high} cells have previously been defined as MAIT cells (Martin et al., 2009; Goldfinch et al., 2010), Tc 17 cells (Northfield et al., 2008; Billerbeck et al., 2010) or memory stem cells (Turtle et al., 2009). Transcriptional profile analysis of different CD161 expressing cells identified a conserved CD161⁺⁺/MAIT cell transcriptional signature enriched in CD8⁺CD161⁺ T cells, which can be extended to CD4⁺CD161⁺ and TCRγδ⁺CD161⁺ T cells (Fergusson et al., 2014). Furthermore, CD161 T cell expressing populations share innate-like, TCR-independent response to interleukin (IL)-12 plus IL-18. This response is independent of regulation by CD161, which acts as a costimulatory molecule in the context of T cell receptor stimulation. Expression of CD161 hence identifies a transcriptional and functional phenotype, shared across human T lymphocytes and independent of both T cell receptor (TCR) expression and cell lineage. The roles of CD8⁺CD161⁺ and CD4⁺CD161⁺ cells have been defined during viral infection (Northfield et al., 2008; Billerbeck et al., 2010; Rowan et al., 2008) and autoimmune diseases (Annibali et al., 2011; Cosmi et al., 2008; Kleinschek et al., 2009), but to date, any role for CD8⁺CD161⁺ cells in cancer biology has not been defined clearly. In the current study, the inventors set out to understand the biology and functional characteristics of CD8⁺CD161⁺ cells.

The inventors have previously reported the functional significance of the murine counterparts of CD161⁺ cells, CD8⁺NK1.1⁺, and have seen elevated numbers of these cells under conditions that mimic viral infection (Konduri et al., 2016).

Microarray analysis of the murine CD8⁺NK1.1⁺ cells revealed a significant upregulation of granzyme production by these cells upon antigenic stimulation in comparison to CD8⁺NK1.1^{neg} counterparts. There was differential expression of innate genes and pathways that play a role in cytotoxic functions. It has been previously reported that the CD161⁺ human equivalents also constitutively express the cytotoxic mediators granzyme B and perforin. In contrast, a quarter of the cells that lack CD161 expression are naïve CD8⁺ T-cells, and even within the memory population express lower levels of granzyme B and perforin (Neelapu et al., 2018). The expression of CCR4 and CCR6 on CD8⁺CD161⁺ cells indicates their ability to maintain tissue residence and home to different organs. Similar expression patterns have been observed in CD161^{high} cells in the circulating blood of MS patients, potentiating their entry into CNS and contributing to the pathogenesis (Annibali et al., 2011). The inventors found that resting CD8⁺CD161^{neg} cells also expressed higher levels of CXCR3, an effector memory marker that leads to CD8⁺ T cell differentiation into short-lived effectors with limited potential for memory (Kurachi et al., 2011).

Although effective against CD19⁺ hematological malignancies, CAR-T cell therapy has been ineffective in targeting solid tumors (Neelapu et al., 2016; Abken, 2015). One principal challenge has been overcoming suppressive signaling by tregs and enhancing effector and memory functions (Klebanoff et al., 2012). Enhancing the persistence of effector and memory T cells can lead to efficient CAR-T cell therapy. In a preclinical model, both CD8⁺ and CD4⁺ subsets expressed synergistic anti-tumor CAR-T activities (Sommermeyer et al., 2016). Similar results were seen in pre-clinical mouse experiments where a combination of engineered CD4⁺ and CD8⁺ T cells induced potent tumor rejection (Moeller et al., 2005; Shedlock and Shen, 2003). Recent clinical trial data on patients with non-Hodgkin lymphoma and chronic lymphocytic leukemia demonstrated the high anti-cancer activity of CD19-CAR-T cells generated from a composition of CD8⁺ and CD4⁺ T cell subsets that were separately expanded in vitro and infused at a ratio of 1:1 (Turtle et al., 2016a). The same result was obtained in clinical trials on patients with B cell acute lymphoblastic leukemia (Turtle et al., 2016b). Another clinical study on patients with high-risk intermediate grade B-lineage non-Hodgkin lymphoma treated either with first generation CD19-CAR-T using isolated CD8⁺ T_{CM} subset or with second generation CD19-CAR-T using both CD8⁺ and CD4⁺ T_{CM} subsets demonstrated the feasibility and safety of both approaches (Turtle et al., 2016c), although the group of CAR-T with CD4⁺ and CD8⁺ T_{CM} and second generation CAR-T cells demonstrated better persistence. These studies highlight the need for evaluating different subsets of T cells and lymphocytes in CAR-T cell therapy. Lymphocyte subsets with inherent killing potential like NK, NKT and γδT cells have been evaluated for CAR potential (Ngai et al., 2018; Liu et al., 2018; Zoon et al., 2015). CD8⁺CD161⁺ cells were earlier defined as effector memory phenotype with less than 1% of CD161^{high} CD8⁺CD45RA^{neg} cells expressing central memory markers CD62L⁺CCR7⁺ (Takahashi et al., 2006). According to previous reports, CD161 negative cells did not alter CD161 expression with anti-CD2, CD3 or CD28 stimulation, and influenza-specific cells did not express CD161 after re-stimulation, even in the presence of cytokines (Northfield et al., 2008) implying that CD161 is not simply a marker of activation and defines a distinct lineage.

Bulk PBMC preparations typically used in CAR T-cell production represent a heterogeneous group of cells which also include highly differentiated antigen experienced subsets. It has been previously reported that naïve (TO, stem cell memory (T_{scm}), and central memory (T_{cm}) subsets for CAR-engineering resulted in more potent anti-tumor responses (Wang et al., 2011; Berger et al., 2008; Gattinoni et al., 2011; Gattinoni et al., 2005). less-differentiated cells may be more beneficial; however, ex vivo culture methods (cytokine composition and culture duration) may promote T-cell differentiation (Alizadeh et al., 2019). The inclusion of IL-7 and IL-15 has been shown to benefit lymphocyte development, differentiation and homeostasis during ex vivo expansion of T cells and results in higher survival in vivo compared to IL-2 expanded CAR-T cells (Xu et al., 2014; Rochman et al., 2009). Some studies have shown that use of IL-7 and IL-15 together may preserve the T_{scm} phenotype and enhance the potency of CAR-T cells (Rochman et al., 2009; Cieri et al., 2013). Ex vivo expansion of CD3/CD28-CAR-T in the presence of IL-7 and IL-15 enhanced the effector activity while retaining a stem/memory potential against GD2 tumor antigen (Gargett et al., 2015). It has also been demonstrated that CAR-T cells expanded with IL-15 preserve stem cell memory phenotype (CD62L⁺CD45RA⁺CCR7⁺). IL-15 also reduced expression of exhaustion markers and increased proliferation upon antigen challenge (Alizadeh et al., 2019). Others have shown that IL-21 promotes expansion of CD2⁺CD28⁺CD8⁺ T cells (Santegoets et al., 2013) and enhances potency of CD19-CAR-T (Rosenberg, 2014). It has been previously reported that addition of IL-15 and IL-21 helps enhance and maintain the memory potential of NKT cells (Ngai et al., 2018). A combination of IL-7, IL-15, and IL-21 based ex vivo expansion of lymphocytes was reported to enhance memory cells, reduce metastasis, and improve survival against murine melanoma (Zoon et al., 2015). In the present study, the inventors found that ex vivo culture and expansion of bulk T cells with a cocktail of IL-7, IL-15, and IL-21 cocktail benefitted primarily the CD8⁺CD161⁺ cell population without significantly altering the phenotypes of bulk PBMC or CD8⁺CD161^{neg} cells grown without IL-21 or even in IL-2 alone.

In summary, the inventors report that CD8⁺CD161⁺ cells and their murine equivalent CD8⁺NK1.1⁺ cells exhibit exceptionally high cytotoxic potential. Gene expression profile analysis by microarray revealed enhanced expression levels of granzyme, perforin, and innate-like receptors by these cells when activated compared to the NK1.1^{neg} and CD161^{neg} counterparts. In vitro, CD8⁺CD161⁺ T-cells killed with greater efficiency than bulk PBMC or CD8⁺CD161^{neg} populations. Use of this subset for T cell-based therapy offers an exciting new opportunity for effective treatment of solid tumors, including PDAC.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### XI. REFERENCES

The following references, to the extent that they provide exemplary procedural or other details supplementary to those set forth herein, are specifically incorporated herein by reference.
U.S. Pat. No. 4,690,915
U.S. Pat. No. 6,225,042
U.S. Pat. No. 6,225,042
U.S. Pat. No. 6,355,479
U.S. Pat. No. 6,355,479
U.S. Pat. No. 6,362,001
U.S. Pat. No. 6,362,001
U.S. Pat. No. 6,410,319
U.S. Pat. No. 6,790,662
U.S. Pat. No. 7,109,304
U.S. Patent Application Publication No. 2009/0004142
U.S. Patent Application Publication No. 2009/0017000
International Publication No. WO2007/103009
Abken, Immunotherapy 7, 535-544, 2015.
Ahmed et al., Cancer Res 67, 5957-5964, 2007.
Ahmed et al., Clin Cancer Res 16, 474-485, 2010.
Ahmed et al., J Clin Oncol 33, 1688-1696, 2015.
Alizadeh et al., Cancer Immunol Res 7, 759-772, 2019.
Altenschmidt et al., 1997.
Annibali et al., Brain 134, 542-554, 2011.
Ansari et al., World J Gastroenterol 21, 3157-3165, 2015.
Assarsson et al., J Immunol 165, 3673-3679, 2000.
Barthel and Goldfeld, 2003.
Beatty et al., Gastroenterology 155, 29-32, 2018.
Berger et al., J Clin Invest 118, 294-305, 2008.
Billerbeck et al., Proc Natl Acad Sci USA 107, 3006-3011, 2010.
Braud et al., Oncoimmunology 7, e1423184, 2018.
Brocker et al., 1998.
Cieri et al., Blood 121, 573-584, 2013.
Cosmi et al., J Exp Med 205, 1903-1916, 2008.
Eshhar et al., 1993.
Eshhar, 1997.
Fergusson et al., Cell Rep 9, 1075-1088, 2014.
Fergusson et al., Front Immunol 2, 36, 2011.
Fergusson et al., Mucosal Immunol 9, 401-413, 2016.
Fitzer-Attas et al., 1998.
Gargett et al., Cytotherapy 17, 487-495, 2015.
Gattinoni et al., J Clin Invest 115, 1616-1626, 2005.
Gattinoni et al., Nat Med 17, 1290-1297, 2011.
Goldfinch et al., Vet Res 41, 62, 2010.
Grada et al., Mol Ther Nucleic Acids 2, e105, 2013.
Havenith et al., Int Immunol 24, 625-636, 2012.
Hegde et al., Mol Ther 21, 2087-2101, 2013.
Heslop et al., 1996.
Hwu et al., 1995.
Kim et al., Nature, Vol. 22(4), pp. 403-410, 2004.
Klebanoff et al., J Immunother 35, 651-660, 2012.
Kleinschek et al., J Exp Med 206, 525-534, 2009.
Konduri et al., Oncoimmunology 5, e1213933, 2016.
Kurachi et al., J Exp Med 208, 1605-1620, 2011.
Liang et al., Front Immunol 8, 1801, 2017.
Liu et al., Leukemia 32, 520-531, 2018.
Marodon et al., 2003.
Martin et al., PLoS Biol 7, e54, 2009.
Maude et al., N Engl J Med 378, 439-448, 2018.
Moeller et al., Blood 106, 2995-3003, 2005.
Moritz et al., 1994.
Neelapu et al., Cancers (Basel) 8, 2016.
Neelapu et al., Immunology, 2018.
Neelapu et al., J Immunol 153, 2417-2428, 1994.
Neelapu et al., Mod Pathol 28, 428-436, 2015.
Neelapu et al., N Engl J Med 377, 2531-2544, 2017.
Neelapu et al., Viral Immunol 18, 513-522, 2005.
Ngai et al., J Immunol 201, 2141-2153, 2018.
Northfield et al., Hepatology 47, 396-406, 2008.
Remington's Pharmaceutical Sciences, 16th Ed., Mack, ed., 1980.
Riddell et al., 1992.
Roberts et al., 1994.
Rochman et al., Nat Rev Immunol 9, 480-490, 2009.
Rosenberg, J Immunol 192, 5451-5458, 2014.
Rowan et al., J Immunol 181, 4485-4494, 2008.
Santegoets et al., J Transl Med 11, 37, 2013.
Schneider, J. Embryol. Exp. Morph, Vol 27, pp. 353-365, 1972.
Seaman et al., J Virol 78, 206-215, 2004.
Shedlock and Shen, Science 300, 337-339, 2003.
Sommermeyer et al., Leukemia 30, 492-500, 2016.
Stancovski et al., 1993.
Takahashi et al., J Immunol 176, 211-216, 2006.
Topalian and Rosenberg, 1987.
Turtle et al., Clin Pharmacol Ther 100, 252-258, 2016b.
Turtle et al., Immunity 31, 834-844, 2009.
Turtle et al., J Clin Invest 126, 2123-2138, 2016a.
Turtle et al., Sci Transl Med 8, 355ra116, 2016c.
van der Stegen et al., Nat Rev Drug Discov 14, 499-509, 2015.
Vera et al., Blood 108, 3890-3897, 2006.
Walter et al., 1995.
Wang et al., Blood 118, 1255-1263, 2011.
Weitjens et al., 1996.
Xu et al., Blood 123, 3750-3759, 2014.
Zoon et al., Int J Mol Sci 16, 8744-8760, 2015.

1. An in vitro or ex vivo method comprising:
(a) obtaining a sample of cells, the sample comprising CD161⁺ T cells; and
(b) culturing the T cells in the presence of IL-7, IL-15 and IL-21,
thereby providing a population of T-cells that are expanded in the number of CD161⁺ cells as compared to non-CD161⁺ cells.

2. The method of claim 1, comprising:
(a) obtaining a sample of cells, the sample comprising CD8⁺CD161⁺ T cells; and
(b) culturing the T cells in the presence of IL-7, IL-15, and IL-21,
thereby providing a population of T-cells that are expanded in the number of CD8⁺CD161⁺ T cells as compared to non-CD8⁺CD161⁺ cells.

3. The method of claim 1, comprising:
(a) obtaining a sample of cells, the sample comprising CD4⁺CD161⁺ T cells; and
(b) culturing the T cells in the presence of IL-7, IL-15, and IL-21,
thereby providing a population of T-cells that are expanded in the number of CD4⁺CD161⁺ T cells as compared to non-CD4⁺CD161⁺ cells.

4. The method of any one of claims 1-3, wherein the cells are further cultured in a media comprising a CD3 and/or CD28 stimulating agent.

5. The method of claim 4, wherein the CD3 and/or CD28 stimulating agent comprises a CD3 and/or CD28-binding antibody.

6. The method of claim 4, wherein the cells are further cultured in a media comprising a CD3-binding antibody, a CD28-binding antibody, Clec2d, and/or a CD161-binding antibody.

7. The method of claim 6, wherein the cells are further cultured in a media comprising about 0.1 to 5.0, 0.3 to 3.0, or 0.5 to 2.0 μg/ml of a CD3-binding antibody, a CD28-binding antibody, Clec2d, and/or a CD161-binding antibody.

8. The method of any one of claims 1-3, comprising:
(a1) obtaining a sample of cells, the sample comprising CD161⁺ T cells;
(a2) culturing the T cells in the presence of a CD3 and/or CD28 stimulating agent and in the presence of IL-7, IL-15, and IL-21; and
(b) culturing the T cells in the presence of IL-7, IL-15, and IL-21,

9. The method of any one of claims 1-3, comprising:
(a1) obtaining a sample of cells, the sample comprising CD161⁺ T cells;
(a2) culturing the T cells in the presence of a CD3-binding antibody, a CD28-binding antibody, a CD161-binding antibody, and/or Clec2d; and in the presence of IL-7, IL-15, and IL-21; and
(b) culturing the T cells in the presence of IL-7, IL-15, and IL-21.

10. The method of claim 9, wherein the culturing step (b) is essentially free of a CD3-binding antibody, a CD28-binding antibody, Clec2d, and/or a CD161-binding antibody.

11. The method of claim 10, wherein the culturing of step (a2) is for about 12 to 72 hours, 24 to 58 hours, or 24 to 36 hours.

12. The method of claim 10, wherein the culturing of step (b) is for at least about 12 hours.

13. The method of claim 12, wherein the culturing of step (b) is for at least about 1, 2, 3, 4, 5, 6, or 7 days.

14. The method of claim 9, wherein the culturing step (b) is essentially free of a CD3-binding antibody and a CD28-binding antibody.

15. The method of any one of claims 1-3, wherein IL-7 is present at about 5-20 ng/ml, IL-15 is present at about 2.5-10 ng/ml, and/or IL-21 is present at about 20-40 ng/ml, such as 10 ng/ml IL-7, 5 ng/ml IL-15, and/or 30 ng/ml IL-21.

16. The method of claim 1 or 15, further comprising purifying or enriching T cells for the presence of CD8⁺CD161⁺ cells in the sample prior to step (b).

17. The method of claim 1 or 15, further comprising purifying or enriching T cells for the presence of CD8⁺CD161⁺ cells in the sample after step (b).

18. The method of claim 16 or 17, wherein enriching T-cells in the sample comprises fluorescent cell sorting, magnetic bead separation, or paramagnetic bead separation.

19. The method of any one of claims 1-18, wherein culturing persists for up to 7, 14, 21, 28, 35, or 42 days.

20. The method of any one of claims 1-19, wherein the culturing is in a serum-containing media.

21. The method of any one of claims 1-19, wherein the culturing is in a serum-free media.

22. The method of any one of claims 1-3, further comprising obtaining the cells from a subject.

23. The method of claim 22, wherein the sample is obtained by apheresis.

24. The method of any one of claims 1-3, wherein the sample is a cryopreserved sample.

25. The method of any one of claims 1-3, wherein the sample is from umbilical cord blood.

26. The method of any one of claims 1-3, wherein the sample is a peripheral blood sample from the subject.

27. The method of any one of claims 1-3, wherein the sample was obtained by apheresis.

28. The method of any one of claims 1-3, wherein the sample was obtained by venipuncture.

29. The method of any one of claims 1-3, wherein the sample comprises a subpopulation of T cells comprising an increased percentage of CD8⁺CD161⁺ cells as compared to a comparable sample as obtained from the subject.

30. The method of any one of claims 1-3, wherein obtaining the sample comprises obtaining the sample from a 3^{rd} party.

31. The method of any one of claims 1-30, further comprising introducing a nucleic acid encoding a chimeric antigen receptor (CAR) or transgenic T-cell receptor (TCR) into a T cell in said sample.

32. The method of claim 31, wherein introducing does not involve infecting or transducing the T cell with a virus.

33. The method of claim 31 or 32, wherein introducing a nucleic acid encoding a CAR or a transgenic TCR into the T cell occurs prior to step (b).

34. The method of claim 31 or 32, wherein introducing a nucleic acid encoding a CAR or a transgenic TCR into the T cell occurs after to step (b).

35. The method of claim 31, wherein the T cell is inactivated for expression of an endogenous T-cell receptor and/or endogenous HLA.

36. The method of claim 31, further comprising introducing a nucleic acid encoding a membrane-bound Cγ cytokine into the T cell.

37. The method of claim 36, wherein the membrane-bound Cγ cytokine is a membrane bound IL-15.

38. The method of claim 36, wherein the membrane-bound Cγ cytokine is an IL-15-IL-15Rα fusion protein.

39. The method of claim 31, wherein culturing comprises culturing the T cells in the presence of dendritic cells or artificial antigen presenting cells (aAPCs).

40. The method of claim 39, wherein the aAPCs comprises a CAR-binding antibody or fragment thereof expressed on the surface of the aAPCs.

41. The method of claim 39, wherein the aAPCs comprise additional molecules that activate or co-stimulate T-cells.

42. The method of claim 40, wherein the additional molecules comprise membrane-bound Cγ cytokines.

43. The method of claim 39, wherein culturing the T cells in the presence of aAPCs comprises culturing the cells at a ratio of about 10:1 to about 1:10 (CAR cells to aAPCs).

44. The method of claim 31, further comprising cryopreserving a sample of the population of transgenic CAR or transgenic TCR cells.

45. The method of claim 31, wherein the CAR or the transgenic TCR is targeted to a cancer-cell antigen.

46. The method of claim 45, wherein the cancer cell antigen is CD19, CD20, ROR1, CD22carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gp120, HIV-1 envelope glycoprotein gp41, GD2, CD123, CD33, CD138, CD23, CD30, CD56, c-Met, meothelin, GD3, HERV-K, IL-11Rα, κ chain, λ chain, CSPG4, ERBB2, EGFRvIII, VEGFR2, HER2-HER3 in combination, or HER1-HER2 in combination.

47. The method of claim 31, wherein the CAR or the transgenic TCR is targeted to a pathogen antigen.

48. The method of claim 47, wherein the pathogen is a fungal, viral, or bacterial pathogen.

49. The method of claim 47, wherein the pathogen is a Plasmodium, trypanosome, Aspergillus, Candida, HSV, HIV, RSV, EBV, CMV, JC virus, BK virus, or Ebola pathogen.

50. The method of any one of claims 1-3, further comprising assessing CD161⁺ T cell content of said sample prior to step (b), after step (b), or both before and after step (b), such as by cell counting/flow cytometry.

51. A T-cell composition made by a method of any one of claims 1-50.

52. A method of providing a T-cell response in a human subject having a disease comprising administering an effective amount of T cells in accordance with claim 31 or 32 to the subject.

53. The method of claim 52, wherein the disease is a cancer and wherein the CAR or the transgenic TCR is targeted to a cancer cell antigen.

54. The method of claim 53, wherein the subject has undergone a previous anti-cancer therapy.

55. The method of claim 54, wherein the subject is in remission or is free of symptoms of the cancer but comprises detectable cancer cells.
